(19) United States
(12) Patent Application Publication
(10) Pub. No.: US 2003/0130205 A1
(43) Pub. Date: Jul. 10, 2003

(54) Novel pharmaceutical anti-infective agents containing carbohydrate moieties and methods of their preparation and use

(76) Inventor: Samuel T. Christian, Alabaster, AL (US)

Correspondence Address: JOHN S. SUNDSMO, BIOMEDPATENT COM, P.O. BOX 535, Vista, CA, 92085, US

(21) Appl. No.: 10/274,798

(22) Filed: Oct. 21, 2002

Related U.S. Application Data
(63) Continuation-in-part of application No. 09/547,506, filed on Apr. 12, 2000.
(63) Continuation-in-part of application No. 09/547,501, filed on Apr. 12, 2000, now abandoned.

(51) Int. Cl. A61K 31/70

(52) U.S. Cl. 514/23; 514/36

(57) ABSTRACT

Hydrophilic N-linked pharmaceutical compositions, methods of their preparation and use in neuraxial drug delivery comprising a glycosyl CNS acting anti-infective prodrug compound covalently N-linked with a saccharide through an amide or an amine bond and a formulary consisting of an additive, a stabilizer, a carrier, a binder, a buffer, an excipient, an emollient, a disintegrant, a lubricating agent, with the proviso that the saccharide moiety is not a cyclodextrin or a glucuronide.

### FIELD OF THE INVENTION

The invention relates generally to compositions and methods for treating infectious disease. This application is a continuation-in-part of U.S. patent application Ser. Nos. 09/547,506 and 09/547,501, both filed on Apr. 12, 2000, and both incorporated herein by reference in their entirety.

### BACKGROUND OF THE INVENTION

Delivery of drugs from the blood into neural tissues (neuraxial delivery), joints and dense connective tissue is a key aspect complicating clinical rehabilitation and intervention techniques. The blood brain barrier and connective tissue barriers effectively limit access of many classes of known and potentially useful pharmaceutical agents.

Development of novel anti-infective agents has recently been rekindled due to emergence of multiple antibiotic resistant bacteria and increased infection-related morbidity and mortality. In 1994 it was reported that of about 40 million patients hospitalized in the United States 2 million patients acquired nosocomial infections and 50 to 60% involved antibiotic resistant bacteria. Infection related morbidity was estimated (at that time) to be about 60-70,000 patients/year (e.g., see Tomasz, A. 1994. New Engl. J. Med. 330 (1): 1248). The death rate from infectious diseases has increased by more than 50% since 1980 (e.g., see New and Reemerging Infectious Diseases. A Global Crisis and Immediate Threat to the Nation's Health. American Society for Microbiology. 1996.) The Director General of the World Health Organization is quoted in the 1996 World Health Report as saying: “We stand on the brink of a global crisis in infectious diseases. No country is safe from them. No country can any longer afford to ignore their threat.”

Anti-infective sulfonyl-amide drugs, of a general class of sulfonilamide drugs, inhibit bacterial, fungal and parasitic growth by inhibiting synthesis of microbial dihydrofolic acid (DHA), i.e., competing para-aminobenzoic acid (PABA). Examples of these agents include, sulfamethoxazole, sulfathiazole, sulfamerazine, sulfadiazine, sulfademethoxine, sulfamethizole, sulfamoxole, sulfapyridine, sulfamethazine, sulfamethoxidiazine, sulfamethoxipyridazine, sulfisomidine and sulfadoxine. Of the latter agents, sulfamethoxazole i.e., N¹-(5-methyl-3-isoxazolyl)sulfanilamide is often used as a faithful backup medication in treatment of penicillin/amoxicillin-resistant infections. To increase potency and decrease toxicity, sulfamethoxazole is often used in combination therapy, for example, in combination with the microbial dihydrofolate reductase inhibitor trimethoprim, i.e., 2,4-diamino-5-(3,4,5-trimethoxybenzyl)pyrimidine (trimethoprim-sulfamethoxazole) or with erythromycin. Combination therapy also reportedly helps counter emergence of bacterial antibiotic resistance. Trimethoprim-sulfamethoxazole formlations are commonly available, e.g. Bactrim™ and Septa™. Unfortunately, both trimethoprim and sulfamethoxazole binding to endogenous plasma proteins, e.g., up to 70% of sulfamethoxazole and 44% of trimethoprim may become protein-associated after oral dosing (PDR, 47^{th} Edition, 1993, p. 833 and p. 1973). The latter protein-binding features may contribute to development of adverse allergic and hypersensitivity reactions in patients. Indicator of a possible underlying adverse reaction include rash, sore throat, fever, arthralgia, cough, sortness of breath, pallor, purpura or jaundice. Adverse reactions include gastrointestinal disturbances (nausea, vomiting, anorexia), allergic skin reactions (rash, urticaria) and severe thrombocytopenia, toxic epidermal necrolysis, fulminant hepatic necrosis, agranulocytosis, aplastic anemia, renal failure and generalized bone marrow supression. As a potential inhibitor of mammalian folic acid metabolism, teratogenicity has been recorded in animal studies with congenital abnormalities suggested in certain retrospective patient studies. Where few good choices for intervention may exist, trimethoprim-sulfamethoxazole has proved useful in treatments of recalcitrant urinary tract, vaginal and middle ear infections, as well as, in treatments of Pneumocystis infections in HIV-infected patients. However, the overt hepatic and liver toxicity limit use of the drug in the elderly and in patients with liver or renal insufficiency. Uses of these drugs are undoubtedly limited by protein-binding, poor aqueous solubility and toxicity.

The ability of both sulfamethoxazole and trimethoprim to evoke allergic and hypersensitivity immune responses has been attributed to formation of complexes with host proteins (e.g., Mauri-Hellweg et al., 1995). However, significantly, it has recently been reported that sulfonilamide drugs may bind directly to antigen receptors of the major histocompatibility complex (von Greyerz et al., 1999), as well as, perhaps to cell surface integrin receptors and fibronectin receptors, such as the platelet gpIIa/IIIb and gpIIb/IIIb receptors involved in thrombosis (Curtis et al., 1994). Sulfonamide anti-infective agents also have poor resistance to ultraviolet light forming free radicals even in crystaline pharmaceutical forms.

Delivery of pharmaceutical agents is often complicated by endogenous mechanisms for recycling, scavenging and transporting mediators and metabolites. For example, tissue enzyme systems exist for altering and inactivating aromatic amines and amides, i.e., including oxioreductases, methylases, acetylases, hdyroxylases and glucuronic acid conjugating enzyme systems. Monoamine oxidases, (e.g. in stomach and intestine), are oxioreductases that deaminate benzylic ring structures with preferential activity for phenylethylamines and benzylamines. O-methyltransferases are enzymes that catalyze addition of a methyl group, usually at the 3 position of a hydroxyl-substituted benzene ring. O-methoxylated derivatives may be further modified by conjugation with glucuronic acid. Glucuronidation of drug metabolites, i.e., involving glucuronosyltransferase and enzyme systems in kidney and intestine may be mechanisms targeting urinary and biliary excretion of phenolic drugs (e.g., see Green et al., 1996). Sulfamethoxazole apparently undergoes N⁴-acetylation and glucuronidation in humans with formation of predominant inactive 1- and 3-oxides and 3′- and 4′-hydroxy derivatives.

Certain cellular mechanisms for transporting glucose are known. For instance, intestinal intracellular transport vesicles containing Na+/glucose co-transporters (SGLTs) are reported to drive active transport of glucose and galactose across the intestinal brush border by harnessing Na+ gradients across the membrane. Net rates of vesicle transport and exocytosis have been estimated to be in the range of 10 thousand to 1 million per second (Wright et al., 1997). Pointing out the essential nature of this transport, missense mutations in SGLT1 result in a potentially lethal inability to transport glucose and galactose (Martin et al., 1996). Specificity's and capabilities of transport are subjects of active current investigation (Mizuma et al., 1994). Antioxidant flavonol compounds, (present in certain foods as glycosides and quercetin glucosides), may be transported across the rat small intestine via a glucose co-transporter pathway (Gee et al., 1998). Intestinal mechanisms for fructose and possible lactose absorption are currently less well understood. Unlike intestinal transport mechanisms, neural glucose transport at the blood brain barrier is reportedly mediated by endothelial cells and the sodium-independent facilitative transporter GLUT1 (Kumagai et al., 1999). At neuronal cells, glucose transport is reportedly mediated predominantly by GLUT3 (Vannucci, S. J. et al., 1998). Neural tissue and dense connective tissues are almost entirely dependent on glucose transport for normal metabolic activity because tissue stores of glucose are low (relative to demand).

Endothelial barriers effectively limit delivery of many pharmaceutically active compounds, including sulfonamide anti-infective agents. Approaches disclosed for delivering drugs to the brain include the following: namely, (i) lipophilic addition and modification of hydrophilic drugs, (e.g., N-methylpyridinium-2-carbaldoxime chloride; 2-PA; U.S. Pat. Nos. 3,929,813 and 3,962,447; Bodor et al, 1976, 1978 and 1981); (ii) linkage of prodrugs to biologically active compounds, (e.g., phenylethylamine coupled to nicotinic acid as modified to form N-methylnicotinic acid esters and amides, Bodor et al., 1981 and 1983; PCT/US83/00725; U.S. Pat. No. 4,540,564); (iii) derivatization of compounds to centrally acting amines (e.g., dihydropyridinium quaternary amine derivatives; PCT/US85/00236); (iv) caging compounds within glycosyl-, maltosyl-, diglucosyl- and dimaltosyl-derivatives of cyclodextrin (Bodor U.S. Pat. No. 5,017,566, issued May 21, 1991; Loftsson U.S. Pat. No. 5,324,718, issued Jun. 28, 1994 disclosing cyclodextrin complexes); and (v) enclosing compounds in cyclodextrin caged complexes (e.g., Yaksh et al., U.S. Pat. No. 5,180,716). However, these approaches suffer from various different disadvantages including poor pharmacokinetic half-life, poor neuraxial bioavailability, variable dosing and side effects.

Objects of the invention provide methods for improved delivery of anti-infective sulfonyl-aminyl and -amidyl glycoconjugates pharmaceutical agents which also have improved physical properties and decreased toxicity.

### SUMMARY OF THE INVENTION

Anti-infective sulfonamide compositions are disclosed as well as methods for their preparation and use in treating infectious diseases. The disclosed agents are hydrophilic prodrug N-linked glycoconjugate sulfonyl-aminyl and -amidyl compounds, having aqueous solubility. The compounds include cyclic and heterocyclic sulfonyl-amidyl and -aminyl anti-infective compounds. Advantageously, the compounds are transportable by saccharide transporters in the gastrointestinal tract and in endothelial cells at tissue and blood brain barriers. Compounds produced according to the methods of the invention find a variety of uses in therapeutic methods for treating infectious diseases.

### DETAILED DESCRIPTION OF THE PREFERRED EMBODIMENT

While it may be relatively common in the pharmaceutical sciences to develop chemical models in an attempt to refine specificity and selectivity of compounds, it is less common to develop models that simultaneously support the needs of two or more receptor-ligand interactions; or, of intracellular transport mechanisms; or, receptor-ligand binding interactions; and/or, increased selective microbicidal activity with decreased patient toxicity. Anti-infective agents for treating infections are known to depend for their pharmaceutical activity upon a complex interplay between anti-microbial specificity, lipophilicity, gastrointestinal bioavailability, and, for central nervous system (CNS) infections, blood-brain barrier penetrability. For use in anti-infective CNS therapies, the active prodrug compound must theoretically be delivered into the CNS or cerebrospinal fluid (CSF) in a relatively intact and active form. Gastrointestinal drug delivery involves problems of transport, metabolism, methylation, acetylation, deamidation, glucuronidation, metabolism and potential for toxicity. Most surprisingly, compositions and methods have been discovered which simultaneously solve the multiple aspects of these most complex delivery problems.

Objects of the invention provide cyclic and heterocyclic sulfonyl-aminyl and -amidyl carbohydrate-linked prodrug anti-infective compounds, as disclosed further in regard to FORMULA I, below. In other objects, the invention provides sulphonyl-amidyl and -aminyl drug compositions having increased therapeutic efficacy at lower administered dosages. In other objects, the invention provides anti-infective sulphonyl-aminyl and -amidyl pharmaceutical compositions comprising lowered effective unit dosage, at which dosage risks of systemic toxicity, allergy and/or hypersensitivity are reduced. In other objects, the invention provides novel therapeutically efficacious pharmaceutical compositions, e.g., tablets, capsules, solutions and the like, employing lower levels of the instant compounds than possible with prior sulfonamide compounds allowing use at lower concentrations with greater efficacy. In other objects, the invention provides modified sulfonyl-amide and -amine drug compositions which lack undesirable binding to MHC proteins, cellular receptors and host proteins thereby providing both increased therapeutic potency and decreased patient risks of developing allergic, hypersensitivity and thrombocytopenic adverse reactions. In other objects, the invention provides compositions, methods of production and uses for timed-release, subcutaneous and intradermal, intranasal, buccal, trouch and suppository forms comprising the instant compounds.

In yet other objects, the invention provides methods for producing hydrophilic sulfonyl-amine and -amide prodrug pharmaceutical agents N-linked to a carbohydrate moiety. In other objects, the invention provides methods for improving the aqueous solubility of poorly soluble sulfonyl-aminyl and -amidyl pharmaceutical agents. In other objects, the invention provides non-toxic multi-dose form compositions and methods for producing them and for using them to treat infectious disease. The instant compounds advantageously have relatively high aqueous solubility, e.g., up to about 4 to about 5 mg/ml, i.e., compared with about 0.04 to about 0.06 mg/ml for sulfamethoxazole. In certain other objects, the invention provides compositions, methods and uses for relatively high therapeutically effective unit doses of N-linked sulfonyl-amidyl and -aminyl prodrug compounds in relatively small volumes. In still other objects, the invention provides therapeutic compositions and methods for delivery of sulfonyl-amine and -amide prodrug compounds lacking a chemically or enzymatically active reactive benzylic amine or amide group. In yet other objects, the invention provides therapeutic compositions and methods for delivery of sulfonanilamide prodrug compounds lacking an N¹ amine nitrogen capable of participating in hydrogen-bonding interactions with serum and tissue proteins. In other objects, the invention provides sulfanilamide for using compounds that are not deamidated by amidases operative in the intestine and stomach.

Objects of the invention also provide methods using the instant compounds in treatments of infectious diseases, and particularly in infectious diseases localized in dense tissues with poor blood supply and/or within the nervous system. In other objects, the invention provides methods for using the instant compounds to treat infections in neurological tissues and in dense tissues with relatively poor blood supply, e.g., connective tissues, tendons and joints.

In yet other objects, the invention provides methods for treating subjects in need thereof, in a manner effective to achieve therapeutic levels of the instant compounds in neuraxial spaces, connective tissues, tendons, ligaments and joints. The latter therapeutic method involves active transport of the instant compounds by endogenous saccharide transporters across the intestinal lumen; then passively transport in blood; followed by active and/or facilitative transport at endothelial barriers, e.g., the blood-brain barrier. In other objects, the invention provides treatment methods for achieving steady-state plasma concentrations in subjects in need thereof using sulfonyl-amide or -amine prodrug compounds of high aqueous solubility. In other objects, the invention provides novel therapeutic methods, not previously possible, occassioned by enhanced delivery and the hydrophilic properties imparted to poorly soluble pharmaceutical agents according to the methods of the invention. In other objects, the invention provides therapeutic methods employing the instant compounds to treat infectious diseases without need to desensitize a hypersensitive or allergic subject. In yet other objects, the invention provides methods for transcutaneous delivery of stable sulfonyl-amide and -amine glycosyl prodrug pharmaceutical compositions, i.e., not possible previously with many prior compounds because of their chemical and UV-light instability.

Abbreviations used herein are as follows: namely, MHC, major histocompatibility complex; CNS, central nervous system; CSF, cerebrospinal fluid; DHA, dihydrofolic acid; PABA, para-aminobenzoic acid; SMX, sulfamethoxazole; STH, sulfathiazole; SMR, sulfamerazine, SDZ, sulfadiazine; SDM, sulfadimethoxine; SID, sulfisomidine; SDX, sulfadoxine; SMT, sulfamethizole; SMO, sulfamoxole; SPD, sulfapyridine; SMZ, sulfamethazine; SMD, sulfamethoxidiazine; and, SMP, sulfamethoxipyridazine.

For purposes of organizing the following disclosure, as well as, improved understanding of the scope and breadth of the subject prodrug compounds which may be used according to the instant therapeutic methods, as well as their constituent structures the subject compounds are generally described by the structure of FORMULA I: as set forth below,

“A—B—D—E”

Formula I

wherein: each of “—” constitutes a single bond; the “A”-moiety constitutes a prodrug; the “B”-moiety constitutes an optional “bridging” alkyl moiety; the “D”-moiety constitutes a nitrogen (N) “linker”; and, the “E”-moiety constitutes a saccharide, as disclosed further below. While certain preferred instant compounds according to FORMULA I are set forth below as representative examples (below), before addressing the specifics, the meanings of general terms relating to FORMULA I are provided as follows: namely,

“Prodrug”, is used interchangeably in reference to the “A-moiety”, FORMULA I (supra), and is disclosed more particularly in regard to FORMULA IV, below, and is intended to mean a pharmaceutical chemical entity active in ameliorating one or more symptoms of a disease in a subject in need thereof. Representative examples are disclosed (below) and illustrated in the EXAMPLES section.

“Bridge”, is used in reference to the B-moiety, of FORMULA I (supra), and intended to mean an optional group according to FORMULA II, below, (as depicted linked through single bonds to each of the A-moiety and the D-moiety, supra):

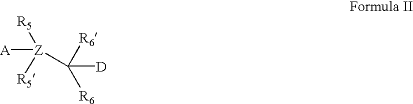

wherein,

Z is optional and when present comprises an optionally R₅- and R_{5′}-substituted lower alkyl; preferably, Z is absent or a lower alkyl comprising 1 or 2 carbon atoms; most preferably, Z is absent or a one carbon atom; and, R₅ and R_{5′} (when present) and R₆ and R_{6′} (when present) are groups selected from among hydrogen, hydroxyl, alkoxyl, carboxyl, alkoxylcarbonyl, aminocarbonyl, alkylamino-carbonyl or dialkylamino-carbonyl.

“Linker”, is used in reference to the D-moiety, FORMULA I (supra), is intended to mean an optionally R₇-substituted amidyl or aminyl nitrogen linking the B-moiety with the E-moiety, i.e., through each of two single bonds, according to FORMULA III, below (depicted linking the B- and E-moieties of FORMULA I):

namely,

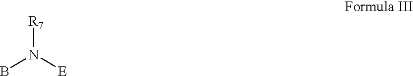

wherein, N comprises a nitrogen atom of a primary or secondary amine or an amide, preferably R₇ is a hydrogen or methyl, most preferably, R₇ is hydrogen.

“Saccharide”, is used in reference to the “E-moiety” of FORMULA I (supra), and is intended to mean a substituted or unsubstituted mono-, di-, tri- or oligosaccharide residue having e.g., constituent sugars comprising 3 carbon atoms (triose), 4 carbons (tetraose), 5 carbons (pentose), 6 carbons (hexose), 7 carbons (heptose), 8 carbons (octose) or 9 carbon atoms (nonose) such as may be present in interrelated straight chain, branched chain and cyclic forms, e.g., in a hexosyl straight chain, furanosyl 5-membered sugar ring, pyranosyl 6-membered sugar ring, and straight and branched oligosaccharide chains composed of monosaccharide sugar residues, as set forth further below.

“Anti-infective prodrug”, when used in regard to the “A” moiety of FORMULA I is intended to mean a sulfonyl-amidyl or sulfonyl-aminyl pharmaceutical agent exerting a growth inhibitory effect on an infectious disease agent, i.e., a microbe as defined below. Representative examples of anti-infective sulfonyl-amidyl prodrug entities include sulfamethoxazole, sulfathiazole, sulfamerazine, sulfadiazine, sulfademethoxine, sulfamethizole, sulfamoxole, sulfapyridine, sulfamethazine, sulfamethoxidiazine, sulfamethoxipyridazine, sulfisomidine, sulfadoxine and the like.

“N-linked glycosyl prodrug”, when used herein in regard to a pharmaceutical agent, is intended to mean an “A”-moiety anti-infective prodrug compound linked through an aminyl or amidyl D-moiety nitrogen to a saccharide E-moiety, according to FORMULA I, supra. Representative N-linked glycosyl prodrug compounds are also disclosed (below) and illustrated (see the EXAMPLES section, below).

“Saccharide” is intended to mean a mono-, di-, tri- or oligosaccharide made up of n sugar subunits linked to each other by glycosidic bonds, which subunits, when n is greater than 1, may be the same or different in respect to the localization of axial and equatorial ring substituents, number of carbon atoms and ring carbon locations and orientations of hydroxyl groups.

“Monosaccharide”, when used in regard to the “E” moiety of FORMULA I, is used interchangeably with sugar to mean a sugar residue. Representative examples of sugar residues include the following: namely, polyhydroxy C₁-aldehydes (e.g. aldoses and ketoaldoses); polyols resulting from e.g., reduction of the C₁ aldehyde carbonyl to a hydroxyl (e.g., alditols and ketoses); polyhdyroxy acids resulting e.g., from oxidation of the C₁ aldehyde and/or the chain terminal hydroxyl (e.g., aldonic, ketoaldonic, aldaric and ketoaldaric); amino-sugars resulting from replacement of any hydroxyl in the chain with an amino group (e.g., aldosamines and ketosamines); aldehydo-acids resulting e.g. from oxidation of only the chain terminal hydroxyl in an aldehydo-sugar (e.g., uronic acids and keto-uronic acids); and their various lactones, i.e., cyclic esters of hydroxy carboxylic acids containing one 1-oxacycloalkan-2-one structure. The subject sugars may be straight chains and/or cyclic 3-, 4-, 5-, 6-, 7-, 8- and 9-membered sugar residues (e.g., hemiacetals and acetals) optionally substituted and linked with the pharmaceutical agent as set forth according to FORMULA I, supra. Representative triosyl residues include the aldoses D- and L-glyceraldehyde and derivatives thereof e.g., glyceraldehyde and glyceric acid phosphates; the keto-sugars D- and L-dihydroxyacetone and derivatives thereof. Representative tetraosyl residues include the aldoses D- and L-erythrose, threose, streptose and apiose; the keto-sugars D- and L-erythrulose; and derivatives thereof. Representative pentosyl residues include the D- and L-aldoses ribose, arabinose, xylose and lyxose; the D- and L-ketoses ribulose and xylulose; and, derivatives thereof. Representative hexosyl residues include aldosyl, furanosyl and pyranosyl sugars, e.g., cyclic and acyclic D- and L-aldoses such as allose, altrose, glucose, mannose, gulose, idose, galactose, talose, fructose, glucono-1,4-lactone, glucaro-1,4:6,3-dilactone, gluconofuranono-6,3-lactone; the ketoses ribo-hexulose, arabino-hexulolose, xylo-hexulose and lyxo-hexulose; and derivatives thereof. Representative 7-membered residues (i.e., heptosyl residues) include e.g., sedoheptulose and derivatives thereof; and, representative 9-membered residues (i.e., nonosyl residues) include N-acetylneuraminic acid and derivatives thereof. Also representative are, 2-deoxy-ribose, 6-deoxyglucose and 2-deoxyglucose, xyloascorbyllactone, digitoxose (2-deoxyaltromethylose), fucose (6-deoxy-galactose), gluconolactone, galaconolactone, rhamnose (6-deoxy-mannose), fructose (2-keto-arabohexose), aldaric acids, alditols, aldonic acids, ketoaldonic acids, and amino sugars; with the proviso that the sugar is not a cyclodextrin. Representative alditols includes e.g., erythritol, threitol, ribitol, arabinitol, xylitol, lyxitol, glucitol, allositol, altrositol, mannositol, gulositol, idositol, galactositol, talositol and their derivatives. Representative aldonic acids include erythronic acid, threonic acid, ribonic acid, arabinonic acid, xylonic acid, lyxonic acid, gluconic acid, allonic acid, altronic acid, mannonic acid, gulonic acid, idonic acid, galactonic acid, tolonic acid and their derivatives. Representative ketoaldonic acids include erythro-tetraulosonic acid, threo-tetraulosonic acid, ribo-pentulosonic acid, arabino-pentulosonic acid, xylo-pentulosonic acid, lyzo-pentulosonic acid, gluco-hexulosonic acid, allo-hexulosonic acid, altro-hexulosonic acid, manno-hexulosonic acid, gulo-hexulosonic acid, ido-hexulosonic acid, galacto-hexulosonic acid, talo-hexulosonic acid and their derivatives. Representative aldaric acids include erythraric acid, threaric acid, ribaric acid, arabinaric acid, xylaric acid, lyxaric acid, allaric acid, altraric acid, glucaric acid, mannaric acid, gularic acid, idaric acid, galactaric acid, talaric acid and their derivatives. Representative of amino sugar include erhtyrosamine, threosamine, ribosamine, arabinosamine, xylosamine, lyxosamine, allosamine, altrosamine, glucosamine, N-acetylglucosamine, N-methlglucosamine mannosamine, gulosamine, idosamine, galactosamine, talosamine and their derivatives. Representative uronic acids include erythrosuronic acid, threosuronic acid, ribosuronic acid, arabinosuronic acid, xylosuronic acid, lyxosuronic acid, allosuronic acid, altrosuronic acid, glucuronic acid, mannosuronic acid, gulosuronic acid, idosuronic acid, galactosuronic acid, talosuronic acid and their derivatives. Representative keto-uronic acids include keto-erythrosuronic acid, keto-threosuronic acid, keto-ribosuronic acid, keto-arabinosuronic acid, keto-xylosuronic acid, keto-lyxosuronic acid, keto-allosuronic acid, keto-altrosuronic acid, keto-glucuronic acid, keto-mannosuronic acid, keto-gulosuronic acid, keto-idosuronic acid, keto-galactosuronic acid, keto-talosuronic acid and their derivatives. Representative lactones include erythrolactone, threolactone, ribolactone, arabinolactone, xyloslactone, lyxoslactone, allolactone, altrolacone, glucolactone, mannolactone, gulolactone, idolactone, galactolactone, talolactone and their derivatives.

Preferred sugar residues for use according to the instant methods comprises aldose or ketose pentosyl or hexosyl sugars selected from the group consisting of D- and L- enantiomers of ribose, glucose, galactose, mannose, arabinose, allose, altrose, gulose, idose, talose and their substituted derivatives. Most preferably, the subject sugar comprises an aldose pentosyl or hexosyl sugar selected from ribose, glucose, galactose, glucosamine, galactosamine, N-acetylglucosamine, N-acetylgalactosamine, N-acetyl ribosamine, xylose, mannose and arabinose.

“Di-saccharide”, when used in regard to the subject sugar residue, is intended to mean a polymeric assemblage of 2 sugar residues. Representative examples of disaccharides include homo-polymeric (e.g., maltose and cellobiose) and hetero-polymeric (e.g., lactose and sucrose) assemblages of sugars as set forth supra.

“Tri-saccharide”, when used in regard to the subject sugar residue, is intended to mean a polymeric assemblage of 3 sugar residues, e.g., as set forth supra.

Preferably, the subject di- and tri-saccharide sugar moieties are metabolizable and/or acid hydrolyzable to mono- and di-saccharides transportable by saccharide transporters in mammals.

“Oligosaccharide”, when used in relation to the subject E-moiety residue of FORMULA I, is intended to mean a polymeric assemblage of about 4 to about 10 glycosidically linked constituent homo-monosaccharide sugars (i.e., all the same constituent) or hetero-monosaccharide (i.e., different constituent) sugars. Each of the subject constituent sugars is linked one-to-another in a serial array through a series of glycosyl bonds formed between the C₁ and C₄ carbon atoms; or alternatively, between the C₁ and C₃ carbon atoms; or alternatively, between the C₁ and C₆ carbon atoms.

The subject oligosaccharides may be homo-polymeric, i.e., all the same sugar constituent, or hetero-monosaccharide, i.e., different constituent sugars. Preferably, the subject oligosaccharide is selected from metabolizable and/or acid hydrolyzable oligosaccharides which following hydrolysis yield mono-, di- and tri-saccharides; and most preferably, the resultant constituent sugars are transportable by a saccharide transporter in a mammal. Representative oligosaccharides include lactose, maltose, isomaltose, sucrose, glycogen, cellobiose, fucosidolactose, lactulose, amylose, fructose, fructofuranose, scillabiose, panose, raffinose, amylopectin, hyaluronic acid, chondroitin sulfate, heparin, laminarin, lichenin and inulin. Preferably, the subject E-moiety, when present as an oligosaccharide, is selected from the group consisting of glucosyl and galactosyl homo- and heteropolymers. Most preferably, the subject E-moiety when present as an oligosaccharide, is selected from the group of metabolizable saccharides consisting of: (i) homopolymers such as an erythran, a threan, a riban, an arabinan, a xylan, a lyxan, an allan, an altran, a glucan (e.g. maltose, isomaltose, cellobiose), a mannan, a gulan, an idan, a galactan, a talan and their substituted derivatives; (ii) heteropolymers such as erythrosides, threosides, ribosides, arabinosides, xylosides, lyxosides, allosides, altrosides, glucosides (e.g., sucrose; (Glc-β1,4Frc), galactosides (e.g., lactose; Gal-β1,4-Glc), mannosides, gulosides, idosides, talosides and their substituted derivatives. Other representative oligosaccharides include the following: namely, sucrose, glycogen, fucosidolactose, lactulose, lactobionic acid, amylose, fructose, fructofuranose, scillabiose, panose, raffinose, amylopectin, hyaluronic acid, chondroitin sulfate, heparin, laminarin, lichenin and inulin. Preferably, the subject sugar, when present as an oligosaccharide, is selected from the group consisting of glucosyl and galactosyl homo- and heteropolymers, e.g., glucans, galactans, glucosides and galactosides. The subject sugar is not a cyclodextrin or derivative thereof. The subject E-moiety is not a cyclodextrin or derivative thereof.

“Aldose” is intended to mean a polyhydroxyaldehyde of the sugar of the general form H[CH(OH)]ₙC(═O)H, wherein n is an integer greater than one; preferably, the subject aldose is in equilibrium with furanosyl and pyranosyl forms.

“Ketose”, also known as ketoaldose, is intended to mean a sugar containing both an aldehydic group and a ketonic carbonyl group; preferably, the subject ketose is in equilibrium with intramolecular hemiacetal forms.

“Aldaric acid” is intended to mean a polyhydroxy dicarboxylic acid of a sugar having the general formula HOC(═O)[CH(OH)]nC(═O)OH, wherein n is greater than 1 and such as may be derived from an aldose by oxidation of both terminal carbon atoms to carboxyl groups.

“Alditol” is intended to mean an acyclic polyol having the general formula HOCH₂[CH(OH)]ₙCH₂OH, wherein n is greater than one.

“Aldonic acid” is intended to mean a polyhydroxy acid having the general formula HOCH₂[CH(OH)]ₙC(═O)OH, wherein n is greater than one and such as may be derived from an aldose by oxidation of the aldehyde function.

“Amino sugar” is intended to mean a sugar (defined supra) having one alcoholic OH group replaced by an amino group.

“Glycosyl” is intended to mean a hexose sugar substituent group; preferably, a glucosyl or galactosyl substituent.

“Glycosylamine”, also known as N-glycosides, is intended to mean glycosyl group attached to an amino —NR₂ group; preferably, an N-linked glucosyl or galactosyl substituent.

“Furanose” is intended to mean a cyclic hemiacetal form of a sugar in which the ring is five membered.

“Pyranose” is intended to mean a cyclic hemiacetal form of a hexose sugar in which the ring is six membered.

As used herein the following additional terms are intended to have meaning as follows: namely,

“Saccharide transporter” is intended to mean a cellular membrane protein capable of binding a saccharide and transporting that saccharide from one location to another on/in the cell. Representative examples of saccharide transporters include a glucose transporters (e.g., GLUT 1, 2, 3, 4 and 5), galactose transporters, a mannose transporters, fructose transporters, arabinose transporters and the like. Those skilled in the art are cognizant of methods by which test compounds may be shown capable of binding to a saccharide transporter, i.e., and examples of which are provided below.

“Pharmaceutical composition”, is intended to mean a composition containing one or more N-linked glycosyl prodrug compounds according to FORMULA I and a formulary effective to provide a dosage form suitable for administration to man or a domestic animal. Representative examples of formularies and dosage forms so suitable are provided below.

“Formulary” is intended to mean an agent added to a pharmaceutical composition comprising said hydrophilic N-linked prodrug compound according to FORMULA I. Representative examples of formulary agents include additives, stabilizers, carriers, binders, buffers, excipients, emollient water-in-oil and oil-in-water emulsions, disintegrants, lubricating agents, antimicrobial agents, preservative and the like; as disclosed further below.

“Dosage form” is intended to mean a form of a pharmaceutical composition suitable for administration to man or a domestic animal. Representative dosage forms include solids and liquids, e.g., perenteral and injection solutions, powders and granules, emollient creams, syrups and elixirs, nasal and ophthalmic drops, intrabronchial inhalants, timed-release capsules, lozenges, troches, suppositories, dermal patches, impregnated bandages and the like.

“Treatment” is intended to mean a method of delivering to a subject in need thereof, i.e., man or a domestic animal, a pharmaceutical preparation with the aim of ameliorating or preventing one or more indicia of an infection in the subject. The subject methods include delivering the preparation to a patient i) before the infection has been diagnosed, (e.g., prophylactic protocols delivered with the aim of preventing development of the infection), as well as, ii) after the infection has been diagnosed, (e.g., therapeutic protocols). That the subject treatments have fulfilled the intended aim will be evident to a skilled practitioner by a change (increase or decrease) or complete elimination of one or more clinical indicia of the infectious disease.

“Indicia of dysfunction” is intended to mean a sign or symptom of an infectious disease as may be evident to a trained professional, e.g., a clinician or specialist, in view of one or more patient clinical symptoms, or in view of a combination of laboratory test results and observations. Representative indicia of infection include clinical symptoms of inflammation, i.e., fever, redness, swelling and the like; neurologic dysfunction resulting from peripheral and central nervous system infection, e.g., motor and sensory dysfunction, disorientation and the like; as well as diagnostic test results and results from microbiological culture and isolation.

“Neurologic dysfunction” is intended to mean a pathophysiologic or psychologic condition of a central or peripheral nervous system tissue, which condition is evidenced by a difference relative to a function of a nervous system activity in a normal healthy control subject. For example, the subject conditions include, but are not limited to, i) toxic dystrophy, (e.g., secondary dystrophy in the nervous system relating to infection), ii) vascular impairment e.g. resulting from infectious and/or inflammatory damage to nervous tissues, iii) central nervous system degeneration or peripheral nerve degeneration, and iv) nervous system lesions induced by infectious agents. Representative illness, diseases, and conditions having neurologic dysfunction have been classified and codified (“International Classification of Diseases, Washington D.C., 1989).

“Subject in need thereof” is intended to mean a mammal, e.g., humans, domestic animals and livestock. Representative examples of subjects in need thereof include humans and domestic animals having an infection. Representative infections include pulmonary infections, nasal infections, bronchial infections, dermal infections, infections of dense tissues, (e.g., muscle, connective tissues, tendons and ligaments), and infections of the peripheral and central nervous system.

“Intestinal cell” is intended to mean a columnar epithelial cell, e.g., a microvillus luminal cell, lining the small or large intestine, or lining the colon.

“Endothelial cell” is intended to mean a cell lining a blood vessel, e.g., a capillary cell or a cell of an artery or a vein.

“Neural cell” is intended to mean cells of the nervous system, including neurons, glial cells, Schwann cells and the like.

“Transportable in an intact form” is intended to mean that the instant N-linked glycosyl prodrug compound is not an inhibitor of a saccharide transporter, and is not substantially chemically altered during transport, e.g., it is not methylated or metabolized to an inactive form or converted to a glucuronide during transport, such that when the instant compound is transported from one side of a cell to the another side it remains substantially chemically and functionally unchanged.

“Neuraxial delivery” is intended to mean that administration of one or more of the instant pharmaceutical compositions, according to FORMULA I, at one or more sites outside the central nervous system results in measurable levels of the A-moiety drug within a neural tissue or a neural tissue fluid. Representative neural tissues include myelinated and non-myelinated nerves, brain and spinal cord. Representative neural tissue fluids include cerebrospinal fluid and tissue homogenates and expressates obtained from myelinated and non-myelinated nerves. Representative methods for measuring levels of the instant prodrugs in biological fluids are known to those of skill in the art.

“Substantially chemically unchanged” means that only conservative modifications of certain R group substituents of the A, B, D or E-moieties (FORMULA I, below) may occur during transport, e.g., removal of a halogen atom and replacement with a hydrogen, conversion of a hydroxyl to a methoxy and the like.

“Brain penetration index”, abbreviated BPI, is intended to mean the mathematical ratio calculated as the amount of one or more of the instant compounds in brain tissue per gram of brain tissue, divided by the amount of the compound (or compounds) in liver tissue per gram liver tissue. The liver being chosen as a reference organ because of its intimate contact with blood and relative lack of barriers. Measurements of BPI may be made for instance at 5-60 minutes after administration of a test compound, e.g., by oral, subcutaneous or intravenous routes. The subject mathematical ratio is commonly expressed as a percentage, i.e., by multiplying the ratio by 100%. This procedure has the advantage that even for a sparingly soluble lipophilic drugs, (which tend to remain largely at an injection site with slow diffusion into the circulation), the amounts of drug in the liver will reflect the actual amount which is systemically available and not the initial dose injected. Certain of the preferred compounds according to the instant invention have BPIs in the range of about 2% to about 500%, most preferred compounds have a BPI of about 10% to about 200%.

“Microbial infection” is intended to mean infection of a mammalian host with a bacteria, virus, fungus, ricketssia, mycoplasma, prion agent, or parasite.

Embodiments of the invention provide pharmaceutical compositions containing a hydrophilic N-linked prodrug compound, according to FORMULA I, and a formulary, preferably in a dosage form as defined supra. The instant N-linked sulfonyl prodrug compounds contain an A-moiety prodrug linked through an aminyl or amidyl bond with a saccharide moiety, preferably a mono-, di- or tri-saccharide. The instant pharmaceutical compositions are suitable for treating a neurological infection in a subject in need thereof without resort to combination therapy, e.g., a treatment with the instant compound an a monoamine oxidase or decarboxylase inhibitor. Despite N-linkage between the subject prodrug compound and the saccharide moiety, the compounds and compositions according to the invention when administered in an oral dosage form are: (i) transportable in a substantially intact form across the gastrointestinal lumen and into blood, i.e., by endogenous active transport mechanisms; then, (ii) transportable in blood to the blood brain barrier (i.e., unassociated or associated with erythrocyte saccharide transporters); and, (iii) transportable across the blood brain barrier into myelinated and unmyelinated neural tissues (i.e., by facilitative transporters in endothelial cells).

In other embodiments, the invention provides methods and processes for preparing a variety of hydrophilic N-linked glycosyl prodrug compounds, each of which methods and processes contains a synthetic step, or series of steps, which result in the formation of an aminyl or an amidyl nitrogen bond between a saccharide moiety and a prodrug compound according to FORMULA I, supra.

In other embodiments, the invention provides processes for preparing pharmaceutical compositions comprising hydrophilic N-linked glycosyl prodrug compounds, according to FORMULA I, suitable for neuraxial delivery. The processes comprise the steps of first linking a prodrug compound, according to FORMULA I, supra, with a saccharide moiety through an aminyl or amidyl nitrogen atom. Representative conditions suitable for formation of amidyl or aminyl nitrogen bonds between the subject prodrug compounds and the saccharide E-moiety are illustrated below. Next, formulary compounds (supra) are added to the resultant N-linked glycosyl prodrug to form the instant pharmaceutical composition. Representative formulary compounds, as disclosed supra, additives, stabilizers, carriers, binders, buffers, excipients, emollients, disintegrants, lubricating agents, antimicrobial agents, preservatives and the like.

In other embodiments, the invention provides methods for treating a subject in need thereof by the step of administering one or more of the instant pharmaceutical compositions comprising an N-linked glycosyl prodrug compound according to FORMULA I to the subject. Preferably, the instant methods involve treatment regimens useful for ameliorating one or more indicia of disease in a subject having an infectious disease, as set forth supra. According to the instant disclosure, pharmaceutical compositions administered according to the instant method provide N-linked glycosyl prodrug compounds which when released from the instant pharmaceutical compositions are transportable across the gastrointestinal tract, transportable in blood, and transportable across the blood brain barrier, or across endothelial barriers in dense tissues, in a substantially intact form. Preferably, within the latter tissue sites the instant N-linked glycosyl prodrug compounds are activatable by a tissue amidase, e.g., a glucosaminidase, a galactosaminidase and the like, to release the A-moiety prodrug from its covalent linkage with the B-D-E-moiety. Most preferably, the A-moiety prodrug, when released, comprises and active drug exerting a growth inhibitory effect on an infectious disease agent, i.e., a microbe.

In yet other embodiments, the invention provides methods for improving the aqueous solubility and blood brain barrier penetrability of a prodrug compound by covalently linking that prodrug compound through an aminyl or amidyl nitrogen bond to a saccharide. In certain preferred embodiments, the subject prodrug compound comprises a prodrug according to FORMULA I, and the instant methods are effective to both increase aqueous solubility and improve blood-brain-penetrability. While it may be common in the art to add hydrocarbon chains to prodrug compounds to increase lipid solubility, (i.e., often at the expense of decreased aqueous solubility), the instant methods provide an alternative, which simultaneously offers advantages of high aqueous solubility and good blood brain barrier penetrability.

In certain presently preferred embodiments, the invention provides methods for administering a anti-infective therapy to a subject in need thereof. The instant method involves administering to the subject one or more of the instant pharmaceutical preparations consisting of an N-linked glycosyl prodrug compound according to FORMULA I, with the requirement that the instant compound, when so administered, is capable of inhibiting the growth of a microbial agent, e.g., inhibiting one or more microbial cellular metabolic processes.

In other embodiments, the invention provides methods for producing prodrug compositions with improved bioavailability, CNS penetrability and adsorption enhancing activity. The methods involve the step (or steps) of linking a saccharide through an amidyl or aminyl nitrogen bond with a sulfonyl prodrug compound to form the instant compound according to FORMULA I.

In certain presently preferred embodiments, the invention provides improved methods for treating infectious diseases. The instant methods employ one or more of the instant N-linked glycosyl prodrug anti-infective compounds (supra), i.e., having improved bioavailability and aqueous solubility, fewer toxic side effects and fewer allergic and hypersensitivity reactions.

In certain other preferred embodiments, the invention provides pharmaceutical compositions containing N-linked glycosyl sulfonyl anti-infective prodrug compounds according to FORMULA I that are effective to inhibit one or more microbial biosynthetic processes at a site of infection at lower dosages than the parent A-moiety (FORMULA I) sulfonyl-amidyl or -aminyl anti-infective drug.

In yet other embodiments, the invention provides anti-infective pharmaceutical compositions with improved aqueous solubility and transportability by saccharide transporters and methods for their use in neuraxial delivery of anti-infective therapy across the intestine (e.g., in timed release dosage forms) and rectum (e.g., in suppositories).

Unlike sulfamethoxazole, presently preferred embodiments of the invention according to FORMULA I provide anti-infective sulfonyl-amidyl and -aminyl prodrug compositions that offer advantages of possible decreased tissue ulceration, irritation and toxicity when injected or applied locally (e.g., onto a skin or mucosal surface) or when delivered into the gastrointestinal lumen via an oral route.

In addition to sulfonyl-amidyl and aminyl prodrugs, the instant methods of the invention find particular other uses for improving the properties of other, (non-sulfonamide), classes of sparingly water-soluble anti-infective prodrug compounds such as may have undesirable toxicological or pharmacokinetic profiles, e.g., trimethoprim. Representative classes of anti-infective pharmaceutical drug compounds that may contain sparingly water soluble, lipophilic and/or water-labile drugs which may prove suitable for use according to the instant methods are disclosed in TABLE A and TABLE B on the following pages. Representative pharmaceutical drug compounds contemplated for improvement according to the instant methods include those set forth in TABLE A, as well as derivatives thereof, with the presently preferred drug compounds disclosed in TABLE B, below.

TABLE A

| Class of Agent: | Representative Examples: |
|---|---|
| Antimicrobial Agents | ampicillin, penicillin G, ketoconazole, itraconazole, metronidazole, miconazole, |
| | co-trimoxazole, amoxicillin, oxacillin, carbenicillin, benzylpenicillin, |
| | phenoxymethylpenicillin, methicillin, nafcillin, ticarcillin, bacampicillin, |
| | epicillin, hetacillin, pivampacillin, the methoxymethyl ester of hetacillin, |
| | ampicillin, chlortetracycline, demeclocycline, minocycline, doxycycline, |
| | oxytetracycline, tetracycline, methacycline, clindamycin, lincomycin, nalidixic |
| | acid, oxolinic acid, phenazopyridine, dicloxacillin, cephalothin, cephalexin, |
| | cefazolin, cefoxitin, moxalactam, ceforanide, cefroxadine, cephapirin, |
| | imidazole-type antifungal agents, econazole, clotrimazole, oxiconazole, |
| | bifonazole, metronidazole (metronidazole benzoate), fenticonazole, miconazole, |
| | sulconazole, tioconazole, isoconazole, butoconazole, ketoconazole, doconazole, |
| | parconazole, orconazole, valconazole and lombazole, trizole-type antifungal |
| | agents, terconazole, itraconazole, co-trimoxazole, sulfadiazine, sulfonamide |
| Antiprotozoal Agents | imidazole-type antiprotozoals, metronidazole, ornidazole, carnidazole, |
| | ipronidazole, tinidazole, nimorazole, benzimidazole-type antifungals, |
| | flubendazole |
| Antihelminthic Agents | benzimidazole-type, thiabendazole, oxibendazole, cambendazole, fenbendazole, |
| | flubendazole, albendazole, oxfendazole |

Presently preferred pharmaceutical A-moiety (FORMULA I) drug compounds according to the methods of the invention are anti-infective drugs of the general class of chemical compounds disclosed in TABLE B, below.

TABLE B
ANTI-MICROBIAL AGENTS:
Sulfamethoxazole

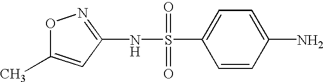

Sulfamethiazole

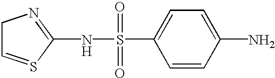

Penciclovir

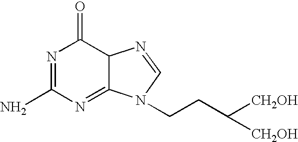

Sulfamerazine

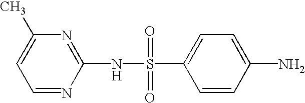

Sulfadiazine

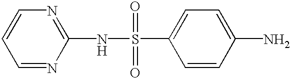

Sulfamethoxine

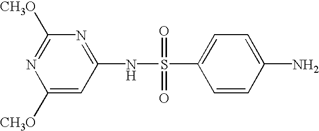

Trimethoprim

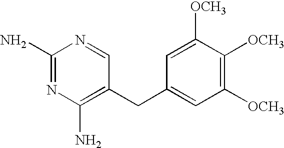

Cephalosporins:
e.g. Cefepime

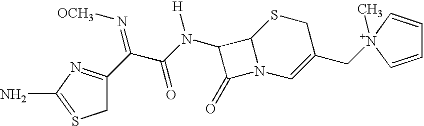

Anti-Fungal Compounds:
e.g. Flucytosine

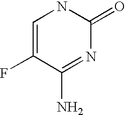

Anti-Parasitic Agents:
e.g. Trimetrexete

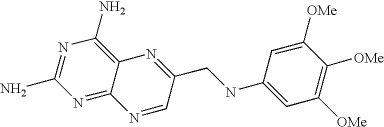

Pentamidine

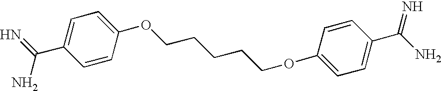

Melarsoporol

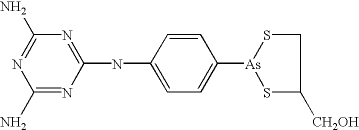

Penicillins:

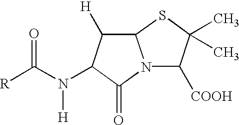

e.g. Amoxicillin

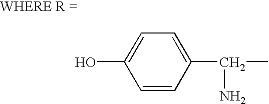

Anti-tuberculosis Agents:
e.g. Ethionamide

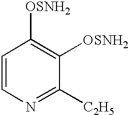

Cycloserine

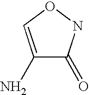

Amino-salicylic acid

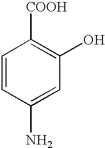

Cycloguanil

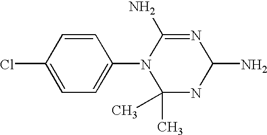

Pyrimethamine

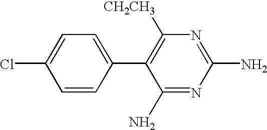

In certain presently preferred embodiments, the A-B-D-E compound of FORMULA I, comprises a compound according to FORMULAS IVa-IVb, wherein, the A-moiety” prodrug is depicted linked through the “B” and “D”-moieties with the “E”-moiety as set forth and described further below: namely,

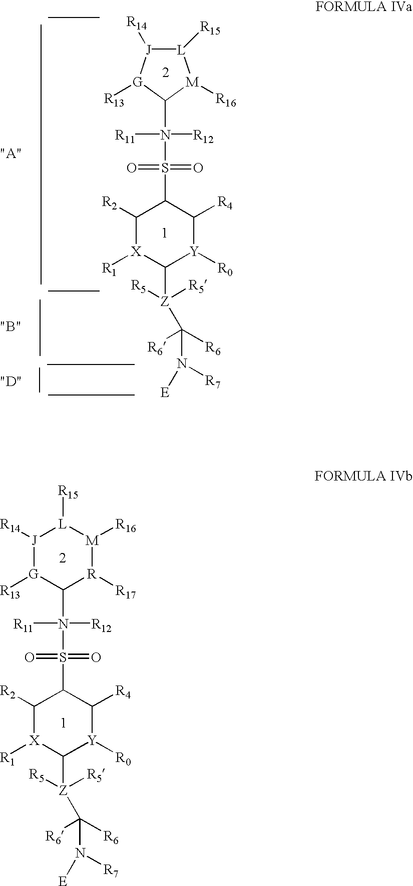

wherein, Ring 1 and Ring 2 each independently comprise an optionally substituted cyclic or heterocyclic ring, or an optionally substituted aromatic ring, Ring 1 being composed of about 4 to about 8 carbon atoms, among which are counted “X” and “Y” and Ring 2 composed of about 4 to about 6 atoms among which are counted “G”, “J”, “L”, “Q” and optional ring components “M” and “R”;

preferably, Ring 1 comprises an optionally substituted aryl or heteroaryl ring and Ring 2 comprises an optionally substituted 5-membered ring or a heteroaryl; and

most preferably, Ring 1 comprises a substituted aryl ring; wherein, R₁, R₂, R₃ and R₄ comprise the subject optional ring substituents and X and Y each comprise a carbon atom;

most preferably Ring 2 comprises an optionally substituted 5-membered ring, i.e., as depicted in FORMULA IVa, or 6-membered ring, i.e., as depicted in FORMULA IVb; and,

wherein the instant 5-membered Ring 2 (FORMULA IVa) is selected from among the compounds numbered 1A through 17E of TABLE C, and the instant 6-membered Ring 2 (FORMULA IVb) is selected from among the compounds numbered 1A through 19F of TABLE D (below).

TABLE C
Alternative Ring 2 Groups: 5-membered

| Compound | | | | | | |
|---|---|---|---|---|---|---|
| Class No. | “G” | “J” | “L” | “M” | “Q” | Representative Ring Strucutres |
| 1A | C | C | N | C | C | pyrrolyl, pyrrolidinyl |
| 1B | C | N | C | C | C | pyrrolyl, pyrrolidinyl |
| 1C | C | C | C | N | C | pyrrolyl, pyrrolidinyl |
| 1D | N | C | C | C | C | pyrrolyl, pyrrolidinyl |
| 1E | C | C | C | C | N | pyrrolyl, pyrrolidinyl |
| 2A | C | C | S | C | C | thiophenyl |
| 2B | C | S | C | C | C | thiophenyl |
| 2C | C | C | C | S | C | thiophenyl |
| 2D | S | C | C | C | C | thiophenyl |
| 2E | C | C | C | C | S | thiophenyl |
| 3A | C | C | O | C | C | furanyl |
| 3B | C | O | C | C | C | furanyl |
| 3C | C | C | C | O | C | furanyl |
| 3D | O | C | C | C | C | furanyl |
| 3E | C | C | C | C | O | furanyl |
| 4A | N | C | N | C | C | imidazolyl, imidazolidinyl |
| 4B | C | N | C | N | C | imidazolyl, imidazolidinyl |
| 4C | C | C | N | C | N | imidazolyl, imidazolidinyl |
| 4D | N | C | C | N | C | imidazolyl, imidazolidinyl |
| 4E | C | N | C | C | N | imidazolyl, imidazolidinyl |
| 5A | C | C | N | N | C | pyrazolyl, pyrazolidinyl |
| 5B | C | N | N | C | C | pyrazolyl, pyrazolidinyl |
| 5C | N | N | C | C | C | pyrazolyl, pyrazolidinyl |
| 5D | C | C | C | N | N | pyrazolyl, pyrazolidinyl |
| 5E | N | C | C | C | N | pyrazolyl, pyrazolidinyl |
| 6A | O | C | N | C | C | oxazolyl, oxazolidinyl |
| 6B | C | O | C | N | C | oxazolyl, oxazolidinyl |
| 6C | C | C | O | C | N | oxazolyl, oxazolidinyl |
| 6D | N | C | C | O | C | SMO: oxazolyl, oxazolidinyl |
| 6E | C | N | C | C | O | oxazolyl, oxazolidinyl |
| 7A | C | N | O | C | C | isooxazolyl, isooxazolidinyl |
| 7B | C | C | N | O | C | isooxazolyl, isooxazolidinyl |
| 7C | C | C | C | N | O | isooxazolyl, isooxazolidinyl |
| 7D | O | C | C | C | N | isooxazolyl, isooxazolidinyl |
| 7E | N | O | C | C | C | SMX: isooxazolyl, isooxazolidinyl |
| 8A | S | C | O | C | C | oxathiolanyl, oxathiolyl |
| 8B | C | S | C | O | C | oxathiolanyl, oxathiolyl |
| 8C | C | C | S | C | O | oxathiolanyl, oxathiolyl |
| 8D | O | C | C | S | C | oxathiolanyl, oxathiolyl |
| 8E | C | O | C | C | S | oxathiolanyl, oxathiolyl |
| 9A | C | S | O | C | C | oxathiolanyl, oxathiolyl |
| 9B | C | C | S | O | C | oxathiolanyl, oxathiolyl |
| 9C | C | C | C | S | O | oxathiolanyl, oxathiolyl |
| 9D | O | C | C | C | S | oxathiolanyl, oxathiolyl |
| 9E | S | O | C | C | C | oxathiolanyl, oxathiolyl |
| 10A | N | C | S | C | C | thiazolidinyl, thiazolyl |
| 10B | C | N | C | S | C | thiazolidinyl, thiazolyl |
| 10C | C | C | N | C | S | thiazolidinyl, thiazolyl |
| 10D | S | C | C | N | C | thiazolidinyl, thiazolyl |
| 10E | C | S | C | C | N | thiazolidinyl, thiazolyl |
| 11A | C | N | S | C | C | isothiazolidinyl, isothiazolyl |
| 11B | C | C | N | S | C | isothiazolidinyl, isothiazolyl |
| 11C | C | C | C | N | S | isothiazolidinyl, isothiazolyl |
| 11D | S | C | C | C | N | isothiazolidinyl, isothiazolyl |
| 11E | N | S | C | C | C | isothiazolidinyl, isothiazolyl |
| 12A | N | C | N | N | C | triazolidinyl, triazolyl |
| 12B | C | N | C | N | N | triazolidinyl, triazolyl |
| 12C | N | C | N | C | N | triazolidinyl, triazolyl |
| 12D | N | N | C | N | C | triazolidinyl, triazolyl |
| 12E | C | N | N | C | N | triazolidinyl, triazolyl |
| 13A | N | C | O | N | C | oxadiazolyl, oxadiazolidinyl |
| 13B | C | N | C | O | N | oxadiazolyl, oxadiazolidinyl |
| 13C | N | C | N | C | O | oxadiazolyl, oxadiazolidinyl |
| 13D | O | N | C | N | C | oxadiazolyl, oxadiazolidinyl |
| 13E | C | O | N | C | N | oxadiazolyl, oxadiazolidinyl |
| 14A | N | C | S | N | C | thiadiazolyl, thiadiazolidinyl |
| 14B | C | N | C | S | N | thiadiazolyl, thiadiazolidinyl |
| 14C | N | C | N | C | S | thiadiazolyl, thiadiazolidinyl |
| 14D | S | N | C | N | C | thiadiazolyl, thiadiazolidinyl |
| 14E | C | S | N | C | N | thiadiazolyl, thiadiazolidinyl |
| 15A | C | N | O | N | C | oxadiazolidinyl, oxadiazolyl |
| 15B | C | C | N | O | N | oxadiazolidinyl, oxadiazolyl |
| 15C | N | C | C | N | O | oxadiazolidinyl, oxadiazolyl |
| 15D | O | N | C | C | N | oxadiazolidinyl, oxadiazolyl |
| 15E | N | O | N | C | C | oxadiazolidinyl, oxadiazolyl |
| 16A | N | C | C | S | C | STX: thiazolyl, thiazolidinyl |
| 16B | C | N | C | C | S | thiazolyl, thiazolidinyl |
| 16C | S | C | N | C | C | thiazolyl, thiazolidinyl |
| 16D | C | S | C | N | C | thiazolyl, thiazolidinyl |
| 16E | C | C | S | C | N | thiazolyl, thiazolidinyl |
| 17A | N | N | C | S | C | SMT: thiadiazolyl, thiadiazolidinyl |
| 17B | C | N | N | C | S | thiadiazolyl, thiadiazolidinyl |
| 17C | S | C | N | N | C | thiadiazolyl, thiadiazolidinyl |
| 17D | C | S | C | N | N | thiadiazolyl, thiadiazolidinyl |
| 17E | N | C | S | C | N | thiadiazolyl, thiadiazolidinyl |

TABLE D
Alternative Ring 2 Groups: Aryl

| Compound | | | | | | | |
|---|---|---|---|---|---|---|---|
| Class No. | “G” | “J” | “L” | “M” | “R” | “Q” | Representative Ring Strucutres |
| 1A | C | C | N | C | C | C | piperidinyl, pyridyl |
| 1B | C | C | C | N | C | C | piperidinyl, pyridyl |
| 1C | C | C | C | C | N | C | piperidinyl, pyridyl |
| 1D | C | C | C | C | C | N | piperidinyl, pyridyl |
| 1E | N | C | C | C | C | C | SPD: piperidinyl, pyridyl |
| 1F | C | N | C | C | C | C | piperidinyl, pyridyl |
| 2A | C | C | S | C | C | C | thiopyranyl |
| 2B | C | C | C | S | C | C | thiopyranyl |
| 2C | C | C | C | C | S | C | thiopyranyl |
| 2D | C | C | C | C | C | S | thiopyranyl |
| 2E | S | C | C | C | C | C | thiopyranyl |
| 2F | C | S | C | C | C | C | thiopyranyl |
| 3A | C | C | O | C | C | C | pyranyl |
| 3B | C | C | C | O | C | C | pyranyl |
| 3C | C | C | C | C | O | C | pyranyl |
| 3D | C | C | C | C | C | O | pyranyl |
| 3E | O | C | C | C | C | C | pyranyl |
| 3F | C | O | C | C | C | C | pyranyl |
| 4A | N | C | N | C | C | C | SDX; SID: pyrimidinyl |
| 4B | C | N | C | N | C | C | pyrimidinyl |
| 4C | C | C | N | C | N | C | SDM: pyrimidinyl |
| 4D | C | C | C | N | C | N | pyrimidinyl |
| 4E | N | C | C | C | N | C | SMR; SDZ; SMD; SMZ: pyrimidinyl |
| 4F | C | N | C | C | C | N | pyrimidinyl |
| 5A | C | C | N | N | C | C | pyridazinyl |
| 5B | C | C | C | N | N | C | pyridazinyl |
| 5C | C | C | C | C | N | N | pyridazinyl |
| 5D | N | C | C | C | C | N | pyridazinyl |
| 5E | N | N | C | C | C | C | SMP: pyridazinyl |
| 5F | C | N | N | C | C | C | pyridazidinyl |
| 6A | N | C | C | N | C | C | piperazinyl |
| 6B | C | N | C | C | N | C | piperazinyl |
| 6C | C | C | N | C | C | N | piperazinyl |
| 6D | N | C | C | N | C | C | piperazinyl |
| 6E | C | N | C | C | N | C | piperazinyl |
| 6F | C | C | N | C | C | N | piperazinyl |
| 7A | O | C | N | C | C | C | oxazinanyl, oxazinyl |
| 7B | C | O | C | N | C | C | oxazinanyl, oxazinyl |
| 7C | C | C | O | C | N | C | oxazinanyl, oxazinyl |
| 7D | C | C | C | O | C | N | oxazinanyl, oxazinyl |
| 7E | N | C | C | C | O | C | oxazinanyl, oxazinyl |
| 7F | C | N | C | C | C | O | oxazinanyl, oxazinyl |
| 8A | N | C | C | O | C | C | oxazinanyl, oxazinyl |
| 8B | C | N | C | C | O | C | oxazinanyl, oxazinyl |
| 8C | C | C | N | C | C | O | oxazinanyl, oxazinyl |
| 8D | O | C | C | N | C | C | oxazinanyl, oxazinyl |
| 8E | C | O | C | C | N | C | oxazinanyl, oxazinyl |
| 8F | C | C | O | C | C | N | oxazinanyl, oxazinyl |
| 9A | S | C | O | C | C | C | oxathianyl, oxathiinyl |
| 9B | C | S | C | O | C | C | oxathianyl, oxathiinyl |
| 9C | C | C | S | C | O | C | oxathianyl, oxathiinyl |
| 9D | C | C | C | S | C | O | oxathianyl, oxathiinyl |
| 9E | O | C | C | C | S | C | oxathianyl, oxathiinyl |
| 9F | C | O | C | C | C | S | oxathianyl, oxathiinyl |
| 10A | C | S | C | S | C | C | dithianyl, dithiinyl |
| 10B | C | C | S | C | S | C | dithianyl, dithiinyl |
| 10C | C | C | C | S | C | S | dithianyl, dithiinyl |
| 10D | S | C | C | C | S | C | dithianyl, dithiinyl |
| 10E | C | S | C | C | C | S | dithianyl, dithiinyl |
| 10F | S | C | S | C | C | C | dithianyl, dithiinyl |
| 11A | S | C | S | N | C | C | dithiazinanyl, dithiazinyl |
| 11B | C | S | C | S | N | C | dithiazinanyl, dithiazinyl |
| 11C | C | C | S | C | S | N | dithiazinanyl, dithiazinyl |
| 11D | C | C | C | S | C | S | dithiazinanyl, dithiazinyl |
| 11E | S | N | C | C | S | C | dithiazinanyl, dithiazinyl |
| 11F | C | S | N | C | C | S | dithiazinanyl, dithiazinyl |
| 12A | N | C | C | S | C | C | thiomorpholinyl, thiazinyl |
| 12B | C | N | C | C | S | C | thiomorpholinyl, thiazinyl |
| 12C | C | C | N | C | C | S | thiomorpholinyl, thiazinyl |
| 12D | S | C | C | N | C | C | thiomorpholinyl, thiazinyl |
| 12E | C | S | C | C | N | C | thiomorpholinyl, thiazinyl |
| 12F | C | C | S | C | C | N | thiomorpholinyl, thiazinyl |
| 13A | C | N | S | C | C | C | thiazinanyl, thiazinyl |
| 13B | C | C | N | S | C | C | thiazinanyl, thiazinyl |
| 13C | C | C | C | N | S | C | thiazinanyl, thiazinyl |
| 13D | S | C | C | C | N | S | thiazinanyl, thiazinyl |
| 13E | S | C | C | C | C | N | thiazinanyl, thiazinyl |
| 13F | N | S | C | C | C | C | thiazinanyl, thiazinyl |
| 14A | N | C | N | N | C | C | triazinanyl, triazinyl |
| 14B | C | N | C | N | N | C | triazinanyl, triazinyl |
| 14C | N | C | N | C | N | N | triazinanyl, triazinyl |
| 14D | N | C | C | N | C | N | triazinanyl, triazinyl |
| 14E | N | N | C | C | N | C | triazinanyl, triazinyl |
| 14F | C | N | N | C | C | N | triazinanyl, triazinyl |
| 15A | N | C | O | N | C | C | oxadiazinanyl, oxadiazinyl |
| 15B | C | N | C | O | N | C | oxadiazinanyl, oxadiazinyl |
| 15C | C | C | N | C | O | N | oxadiazinanyl, oxadiazinyl |
| 15D | N | C | C | N | C | O | oxadiazinanyl, oxadiazinyl |
| 15E | O | N | C | C | N | C | oxadiazinanyl, oxadiazinyl |
| 15F | C | O | N | N | C | N | oxadiazinanyl, oxadiazinyl |
| 16A | N | C | S | N | C | C | thiadiazinanyl, thiadiazinyl |
| 16B | C | N | C | S | N | C | thiadiazinanyl, thiadiazinyl |
| 16C | C | C | N | C | S | N | thiadiazinanyl, thiadiazinyl |
| 16D | N | C | C | N | C | S | thiadiazinanyl, thiadiazinyl |
| 16E | S | N | C | C | N | C | thiadiazinanyl, thiadiazinyl |
| 16F | C | S | N | C | C | N | thiadiazinanyl, thiadiazinyl |
| 17A | C | N | O | N | C | C | oxadiazinanyl, oxadiazinyl |
| 17B | C | C | N | O | N | C | oxadiazinanyl, oxadiazinyl |
| 17C | C | C | C | N | O | N | oxadiazinanyl, oxadiazinyl |
| 17D | N | C | C | C | N | O | oxadiazinanyl, oxadiazinyl |
| 17E | O | N | C | C | C | N | oxadiazinanyl, oxadiazinyl |
| 17F | N | O | N | C | C | C | oxadiazinanyl, oxadiazinyl |
| 18A | N | N | C | S | C | C | thiadiazinanyl, thiadiazinyl |
| 18B | C | N | N | C | S | C | thiadiazinanyl, thiadiazinyl |
| 18C | C | C | N | N | C | S | thiadiazinanyl, thiadiazinyl |
| 18D | S | C | C | N | N | C | thiadiazinanyl, thiadiazinyl |
| 18E | C | S | C | C | N | N | thiadiazinanyl, thiadiazinyl |
| 18F | N | C | S | C | C | N | thiadiazinanyl, thiadiazinyl |

R₀, R₁, R₂, R₃ and R₄ each independently comprise a group selected from among hydrogen, hydroxyl, halogen, halo-lower alkyl, alkoxy, alkoxy-lower alkyl, halo-alkoxy, thioamido, amidosulfonyl, alkoxycarbonyl, carboxamide, amino-carbonyl, and alkylamine-carbonyl; preferably, each comprises a group selected from hydrogen, hydroxyl, lower alkyl and alkoxyl-lower alkyl; most preferably, each comprises hydrogen;

R₁₃, R₁₄, R₁₅, R₁₆ each independently comprise a group selected from among hydrogen, hydroxyl, lower alkyl and alkoxyl-lower alkyl; preferably, each is independently hydrogen or lower alkyl; and, most preferably, each is independently hydrogen or lower alkyl;

Z is optional and when present comprises a lower alkyl optionally substituted with R₅ and R_{5′}; preferably, Z is absent or a lower alkyl comprising 1 or 2 carbon atoms; most preferably, Z is absent or a one carbon atom; and, R₅ and R_{5′} (when present) and R₆ and R_{6′} (when present) are groups selected from among hydrogen, hydroxyl, alkoxyl, carboxyl, alkoxylcarbonyl, aminocarbonyl, alkylamino-carbonyl and dialkylamino-carbonyl;

N comprises a nitrogen atom of a primary or secondary amine or an amide, preferably R₇ is a hydrogen or methyl, most preferably, R₇ is hydrogen; and,

E comprises a saccharide moiety as set forth above and below.

Representative examples of E-moiety saccharide residues include the following: namely, polyhydroxy C₁ aldehydes (e.g. aldoses and ketoaldoses); polyols resulting from e.g., reduction of the C₁ aldehyde carbonyl to a hydroxyl (e.g., alditols and ketoses); polyhdyroxy acids resulting e.g., from oxidation of the C₁ aldehyde and/or the chain terminal hydroxyl (e.g., aldonic, ketoaldonic, aldaric and ketoaldaric); amino-sugars resulting from replacement of any hydroxyl in the chain with an amino (e.g., aldosamines and ketosamines); aldehydo- acids resulting e.g. from oxidation of only the chain terminal hydroxyl in an aldehydo-sugar (e.g., uronic acids and keto-uronic acids); and their various lactones, i.e., cyclic esters of hydroxy carboxylic acids containing one 1-oxacycloalkan-2-one structure. The subject sugars may be straight chains and/or cyclic 3-, 4-, 5-, 6-, 7-, 8- and 9-membered sugar residues (e.g., hemiacetals and acetals) optionally substituted and linked with the D-moiety as set forth, supra. Representative triosyl residues include the aldoses D- and L-glyceraldehyde and derivatives thereof e.g., glyceraldehyde and glyceric acid phosphates; the keto-sugars D- and L-dihydroxyacetone and derivatives thereof. Representative tetraosyl residues include the aldoses D- and L-erythrose, threose, streptose and apiose; the keto-sugars D- and L-erythrulose; and derivatives thereof. Representative pentosyl residues include the D- and L-aldoses ribose, arabinose, xylose and lyxose; the D- and L-ketoses ribulose and xylulose; and, derivatives thereof. Representative hexosyl residues include aldosyl, furanosyl and pyranosyl sugars, e.g., cyclic and acyclic D- and L-aldoses such as allose, altrose, glucose, mannose, gulose, idose, galactose, talose, fructose, glucono- 1,4-lactone, glucaro-1,4:6,3-dilactone, gluconofuranono-6,3-lactone; the ketoses ribo-hexulose, arabino-hexulolose, xylo-hexulose and lyxo-hexulose; and derivatives thereof. Representative 7-membered residues (i.e., heptosyl residues) include e.g., sedoheptulose and derivatives thereof; and, representative 9-membered residues (i.e., nonosyl residues) include N-acetylneuraminic acid and derivatives thereof. Also representative are, 2-deoxy-ribose, 6-deoxyglucose and 2-deoxyglucose, xyloascorbyllactone, digitoxose (2-deoxyaltromethylose), fucose (6-deoxy-galactose), gluconolactone, galaconolactone, rhamnose (6-deoxy-mannose), fructose (2-keto-arabohexose), aldaric acids, alditols, aldonic acids, ketoaldonic acids, and amino sugars; with the proviso that the E-moiety is not a cyclodextrin. Representative alditols include e.g., erythritol, threitol, ribitol, arabinitol, xylitol, lyxitol, glucitol, allositol, altrositol, mannositol, gulositol, idositol, galactositol, talositol and their derivatives. Representative aldonic acids include erythronic acid, threonic acid, ribonic acid, arabinonic acid, xylonic acid, lyxonic acid, gluconic acid, allonic acid, altronic acid, mannonic acid, gulonic acid, idonic acid, galactonic acid, tolonic acid and their derivatives. Representative ketoaldonic acids include erythro-tetraulosonic acid, threo-tetraulosonic acid, ribo-pentulosonic acid, arabino-pentulosonic acid, xylo-pentulosonic acid, lyxo-pentulosonic acid, gluco-hexulosonic acid, allo-hexulosonic acid, altro-hexulosonic acid, manno-hexulosonic acid, gulo-hexulosonic acid, ido-hexulosonic acid, galacto-hexulosonic acid, talo-hexulosonic acid and their derivatives. Representative aldaric acids include erythraric acid, threaric acid, ribaric acid, arabinaric acid, xylaric acid, lyxaric acid, allaric acid, altraric acid, glucaric acid, mannaric acid, gularic acid, idaric acid, galactaric acid, talaric acid and their derivatives. Representative of amino sugar include erhtyrosamine, threosamine, ribosamine, arabinosamine, xylosamine, lyxosamine, allosamine, altrosamine, glucosamine, N-acetylglucosamine, N-methlglucosamine mannosamine, gulosamine, idosamine, galactosamine, talosamine and their derivatives. Representative uronic acids include erythrosuronic acid, threosuronic acid, ribosuronic acid, arabinosuronic acid, xylosuronic acid, lyxosuronic acid, allosuronic acid, altrosuronic acid, glucuronic acid, mannosuronic acid, gulosuronic acid, idosuronic acid, galactosuronic acid, talosuronic acid and their derivatives. Representative keto-uronic acids include keto-erythrosuronic acid, keto-threosuronic acid, keto-ribosuronic acid, keto-arabinosuronic acid, keto-xylosuronic acid, keto-lyxosuronic acid, keto-allosuronic acid, keto-altrosuronic acid, keto-glucuronic acid, keto-mannosuronic acid, keto-gulosuronic acid, keto-idosuronic acid, keto-galactosuronic acid, keto-talosuronic acid and their derivatives. Representative lactones include erythrolactone, threolactone, ribolactone, arabinolactone, xyloslactone, lyxoslactone, allolactone, altrolacone, glucolactone, mannolactone, gulolactone, idolactone, galactolactone, talolactone and their derivatives.

Preferably, the subject E-moiety comprises an aldose or ketose pentose or hexose sugar selected from the group consisting of D- and L- enantiomers of ribose, glucose, galactose, mannose, arabinose, allose, altrose, gulose, idose, talose and their substituted derivatives. Most preferably, the subject E-moiety comprises an aldose pentosyl or hexosyl sugar selected from ribose, glucose, galactose, glucosamine, galactosamine, N-acetylglucosamine, N-acetylgalactosamine, N-acetyl ribosamine, xylose, mannose and arabinose.

“Halogen” is intended to mean a fluorine, chlorine, bromine, or sulfur atom or ion or group. Preferred halo groups are chlorine, bromine, thiol and sulfonyl and most preferred, chlorine.

“Lower alkyl” is intended to mean a hydrocarbon chain containing fewer than six carbon atoms, preferably fewer than four and most preferably two or 3 carbon atoms. Representative lower alkyl groups include methyl, ethyl, n-propyl, i-propyl, n-butyl, t-butyl and i-butyl. Presently preferred alkyls are methyl, ethyl or i-propyl, and most preferably, ethyl.

“Substituted lower alkyl” is intended to mean a lower alkyl in which one or more of the hydrogen atoms are replaced by a substituent group. Representative substituent groups include hydroxy, alkoxy, halogen, amino, amido, carboxyl, thiol, sulfonyl, methoxy and the like.

“Halo-lower alkyl” is intended to mean a lower alkyl in which one or more of the hydrogen atoms on the hydrocarbon chain has been replaced by a halogen atom.

“Cycloalkyl” is intended to mean a closed saturated monocyclic hydrocarbon ring made up of about 4 to about 9 carbon atoms, preferably about 5 to about 7 carbon atoms and most preferably 6 carbon atoms. Representative examples of cycloalkyl compounds include phenyl, piperidyl, piperazinyl, diazinyl, morpholinyl, isooxazoanyl and the like.

“Heterocyclic” is intended to mean a close saturated monocyclic ring made up of about 4 to about 8 carbon atoms and about 1 to about 2 non-carbon atoms; preferably, about 5 to about 6 carbon atoms and 1 non-carbon halogen or oxygen atom; and, most preferably 5 carbon atoms and 1 non-carbon halogen or oxygen atom.

“Aromatic”, and “aryl”, are used interchangeably to mean a closed unsaturated monocyclic hydrocarbon ring system made up of about 3 to about 9 carbon atoms having a delocalized π-electron system. Preferably, the subject aryl ring is made up of about 5 to about 7 carbon atoms and most preferably, 6 carbon atoms. Representative aromatic rings include benzyl, pyranyl, pyridyl, pyrimidinyl, thiadiazinyl and pyridazinyl, with benzyl preferred.

“Amine” is intended to mean an —NHR substituent group.

“Amide” is intended to mean an —C(O)N—(R′)R″ or —HNC(O) substituent group, where R′ and R″ are hydrogen or a substituent such as hydroxy, lower alkyl, amino, or the like. Preferred amino groups are those wherein R′ or R″ is hydrogen.

“Alkoxy” is intended to mean an —OR substituent group.

“Halo-lower alkyl” is intended to mean a halogen substituted lower alkyl; preferably, a halogen substituted lower alkyl having 2 to 6 carbon atoms; most, preferably, a chlorine or fluorine substituted lower alkyl having 2 to 4 carbon atoms.

“Alkoxy-lower alkyl” is intended to mean an alkoxy compound, supra, wherein R comprises a lower alkyl; preferably a 2 to 6 carbon lower alkyl; and most preferably, a 2 to 4 carbon lower alkyl.

“Thioalkoxy” is intended to mean an —SOR substituent group.

“Aminocarbonyl” is intended to mean a —C(O)NH₂ substituent group.

“Alkylaminocarbonyl” is intended to mean a —C(O)NHR substituent group wherein R is a lower alkyl.

“Alkoxycarbonyl” is intended to mean a —C(O)OR substituent group.

“Carboxamide” is intended to mean a —NR′COR substituent group.

“Dialkylaminocarbonyl” is intended to mean a —C(O)NR′R substituent group, wherein R′ and R constitute lower alkyl groups.

“Haloalkoxy” is intended to mean a —OR substituent group where R is a haloalkyl.

“Oxyamido” is intended to mean a —OC(O)NH— or —HNC(O)O— substituent.

“Thioamido” is intended to mean a —SC(O)NH— or —HNC(S)— substituent.

“Amidosulfonyl” is intended to mean a —NHSO₂— substituent.

In other embodiments, the invention provides methods of using pharmaceutical compositions containing one or more compounds according to FORMULA I, supra, in combination with optional stabilizers, carriers, binders, buffers, excipients, emollients, disintegrants, lubricating agents, antimicrobial agents and the like. For oral administration, the instant methods may employ pharmaceutical compositions that are liquid, solid or encapsulated. For perenteral administration, the instant methods may employ pharmaceutical compositions that are sterile liquids or solids, e.g., as provided in a powdered or granulated form suitable for reconstitution.

In other embodiments, the invention provides methods of using pharmaceutical compositions containing one or more compounds according to FORMULA I, supra, in combination with an optional second anti-infective agent, and also optional stabilizers, carriers, binders, buffers,′ excipients, emollients, disintegrants, lubricating agents, antimicrobial agents and the like. For oral administration, the instant methods may employ pharmaceutical compositions that are liquid, solid or encapsulated. For perenteral administration, the instant methods may employ pharmaceutical compositions that are sterile liquids or solids, e.g., as provided in a powdered or granulated form suitable for reconstitution. In one presently preferred embodiment one compound according to FORMULA I, supra, is administered in an admixed formulation with the anti-infective drug trimethoprim.

The instant methods may employ compounds to be administered alone or in combination with pharmaceutically acceptable carriers, e.g. in either single or multiple doses. Suitable pharmaceutical carriers may include inert solid diluents or fillers, sterile aqueous solutions, and various nontoxic organic solvents. The pharmaceutical compositions formed by combining a compound according to FORMULA I with a pharmaceutically acceptable carrier may be administered according to the instant methods in a variety of dosage forms such as tablets, lozenges, syrups, injectable solutions, and the like. The subject pharmaceutical carriers can, if desired, contain additional ingredients such as flavorings, binders, excipients, and the like. Thus, for purposes of the instant oral administration, tablets containing various excipients such as sodium citrate, calcium carbonate, and calcium phosphate may be employed along with various disintegrants such as starch, and preferably potato or tapioca starch, alginic acid, and certain complex silicates, together with binding agents such as polyvinylpyrolidone, sucrose, gelatin, and acacia. Additionally, lubricating agents, such as magnesium stearate, sodium lauryl sulfate, and talc may be useful for tableting purposes. Solid compositions of a similar type may also be employed as fillers in salt and hard-filled gelatin capsules. Preferred materials for this purpose include lactose or milk sugar and high molecular weight polyethylene glycols. When aqueous suspensions of elixirs are desired for oral administration according to the instant methods, the compound therein may be combined with various sweetening or flavoring agents, colored matter or dyes, and if desired, emulsifying or suspending agents, together with diluents such as water, ethanol, propylene glycol, glycerin, and combinations thereof. For parenteral administration according to the instant methods, solutions may be prepared in sesame or peanut oil or in aqueous polypropylene glycol, as well as sterile aqueous saline solutions of a corresponding water-soluble pharmaceutically acceptable metal salt, e.g. as disclosed supra. The subject aqueous solution is preferably suitably buffered if necessary and the liquid diluent first rendered isotonic with sufficient saline or glucose. Such aqueous solutions of compounds according to FORMULA I may be particularly suitable for intravenous, intramuscular, subcutaneous, and intraperitoneal injection. The subject sterile aqueous media employed are obtainable by standard techniques well known to those skilled in the art.

For use in one or more of the instant methods, it may prove desirable to stabilize a compound according to FORMULA I, e.g. to increase shelf life and/or pharmacokinetic half-life. Shelf-life stability may be improved by adding excipients such as: a) hydrophobic agents (e.g., glycerol); b) non-linked sugars (e.g., sucrose, mannose, sorbitol, rhamnose, xylose); c) non-linked complex carbohydrates (e.g., lactose); and/or d) bacteriostatic agents. For use in the instant methods, pharmacokinetic half-lives may vary depending upon the saccharide moiety selected, e.g., whether a sugar or a digestible oligosaccharide, or the nature of the sugar R-group constituents. For use in the instant methods, pharmacokinetic half-life and pharmacodynamics may also be modified e.g. by: a) encapsulation; b) controlling the degree of hydration; and, c) controlling the electrostatic charge and hydrophobicity of the sugar constituents.

For use according to the instant methods, pharmaceutically acceptable salts can be prepared from the instant compounds by conventional methods. Thus, such salts may, for example, be prepared by treating a compound according to FORMULA I with an aqueous solution of the desired pharmaceutically acceptable metallic hydroxide or other metallic base and evaporating the resulting solution to dryness, preferably under reduced pressure in a nitrogen atmosphere. Alternatively, a solution of the subject compound may be mixed with an alkoxide to the desired metal, and the solution subsequently evaporated to dryness. The pharmaceutically acceptable hydroxides, bases, and alkoxides include those with cations for this purpose, including (but not limited to), potassium, sodium, ammonium, calcium, and magnesium. Other representative pharmaceutically acceptable salts include hydrochloride, hydrobromide, sulfate, bisulfate, acetate, oxalate, valarate, oleate, laurate, borate, benzoate, lactate, phosphate, tosulate, citrate, maleate, furmarate, succinate, tartrate, and the like.

For use in the instant methods, freely-soluble salts of a compound according to FORMULA I may be converted to a salt of a lower solubility in a body fluid, e.g. by modification with a slightly water-soluble pharmaceutically acceptable salt such as tannic or palmoic acid, or by inclusion in a time-release formulation such as covalently coupled to a larger carrier, or in timed-release capsules and the like. In general, the acid addition salts of the subject compounds with pharmaceutically acceptable acids will be biologically equivalent to the compounds themselves. Pharmaceutically acceptable salts can be prepared from the compounds by conventional methods. Thus, such salts are, for example, prepared by treating with an aqueous solution of the desired pharmaceutically acceptable metallic hydroxide or other metallic base and evaporating the resulting solution to dryness, preferably under reduced pressure in a nitrogen atmosphere. Alternatively, a solution of a compound is mixed with an alkoxide to the desired metal, and the solution subsequently evaporated to dryness. The pharmaceutically acceptable hydroxides, bases, and alkoxides include those with cations for this purpose, including (but not limited to), potassium, sodium, ammonium, calcium, and magnesium. Other representative pharmaceutically acceptable salts include hydrochloride, hydrobromide, sulfate, bisulfate, acetate, oxalate, valarate, oleate, laurate, borate, benzoate, lactate, phosphate, tosylate, citrate, maleate, furmarate, succinate, tartrate, and the like.

The preferred pharmaceutical compositions for inocula and dosage for use in the instant methods will vary with the clinical indication. The inocula may typically be prepared from a dried compound by suspending the compound in a physiologically acceptable diluent such as water, saline, or phosphate-buffered saline. Some variation in dosage will necessarily occur depending upon the condition of the patient being treated, and the physician will, in any event, determine the appropriate dose for the individual patient. The effective amount of the instant compound per unit dose depends, among other things, on the body weight, physiology, and chosen inoculation regimen. A unit dose of a compound according to FORMULA I refers to the weight of the subject compound without the weight of carrier (when carrier is used). Generally, the amount of active ingredient administered to a subject in need thereof according to the practice of the invention will be in the range of about 1 mg/day to about 2.5 gm/day. Single unit dosage forms and multi-use dosage forms are considered within the scope of the invention, as disclosed further below.

Pharmaceutically acceptable carriers may be formed, filled and sealed for ease of use according to the methods of the invention. Representative forming, filling and sealing methods are known in the pharmaceutical arts. For instant, the subject compositions may be formulated with pharmaceutically acceptable carriers into pharmaceutical preparations suitable for inclusion in timed-release capsules, tablets, lozenges, syrups and the like.

For treatments of local infections, the subject compounds may be provided in an emollient cream. Representative examples of emollient pharmaceutically acceptable carriers include oil-in-water and water-in-oil emulsions, i.e., as are known to those skilled in the pharmaceutical arts.

Pharmaceutically acceptable salts may be prepared from the subject compounds by conventional methods. For example, such salts may be prepared by treating one or more of the subject compounds with an aqueous solution of the desired pharmaceutically acceptable metallic hydroxide or other metallic base and evaporating the resulting solution to dryness, preferably under reduced pressure in a nitrogen atmosphere. Alternatively, a solution of the subject compound may be mixed with an alkoxide to the desired metal, and the solution subsequently evaporated to dryness. The pharmaceutically acceptable hydroxides, bases, and alkoxides include those with cations for this purpose, including (but not limited to), potassium, sodium, ammonium, calcium, and magnesium. Other representative pharmaceutically acceptable salts include hydrochloride, hydrobromide, sulfate, bisulfate, acetate, oxalate, valarate, oleate, laurate, borate, benzoate, lactate, phosphate, tosulate, citrate, maleate, furmarate, succinate, tartrate, and the like.

In alternative embodiments, the invention provides different routes for delivery of compounds according to FORMULA I as may be suitable for use in the different infectious disease states and sites where treatment is required. For topical, intrathecal, intramuscular or intra-rectal application it may prove desirable to apply the subject compounds as a salve, ointment or emollient pharmaceutical composition at the local site, or to place an impregnated bandage or a dermal timed-release lipid-soluble patch. For intra-rectal application it may prove desirable to apply the subject compounds e.g. in a suppository. In other embodiments, it may prove desirable to administer the subject compositions by intranasal or intrabronchial instillation (e.g., as pharmaceutical compositions suitable for use in a nebulizer), or by gastrointestinal delivery (e.g., with a capsule, tablet, trouch or suppository). Also contemplated are suppositories for urethral and vaginal use. In one preferred embodiment, the subject pharmaceutical compositions are administered via suppository taking advantage of saccharide transporters in the rectum for transport into the blood stream in a timed-release type manner e.g. providing possible anti-infective therapy in a patient with an immunodeficiency syndrome and a Pneumocystis carinii infection.

Embodiments of the invention provide treatments for infectious diseases with several different microbes including e.g., Pseudomonas aeruginosa, Escherichia coli, Klebsiella species, Enterobacter species, Morganella morganii, Proteus mirabilis, Proteus vulgaris, Haemophilus influenzae, Streptococcus, Shigella flexneri, Shigella sonnei, Shigella dysenteriae, Pneumocystis carinii and antibiotic resistant strains thereof. Infections that may be amenable to treatments according to the instant invention include, e.g., pulmonary infections (pneumonia, chronic bronchitis, infections in cystic fibrosis patients, Pneumocystis carinii infections in HIV infected patients and the like); urinary tract infections; vaginal infections; middle ear infections (otitis media); gastrointestinal infections (e.g., shigellosis, enterotoxic E. coli enteritis and the like; central and peripheral nervous system infections; and, infections of dense tissues, e.g., connective tissues, tendons, ligaments and the like.

In yet other embodiments, the invention provides therapeutic methods in which a relatively high concentration of active ingredients (e.g., up to about 4 to about 5 mg/ml) is included in a relatively small volume taking advantage of the special aqueous solubility of the prodrug compounds according to FORMULA I. In certain embodiments, the invention provides improved treatment methods using relatively high concentrations of the subject drugs in multi-dose, time-release, subcutaneous and intradermal, buccal, trouch, and suppository preparations. In other embodiments, the instant treatment methods may also be especially useful for achieving steady state plasma levels in subjects in need thereof. Where conventional methods of administration may be ineffective in certain patients, the instant methods, i.e., employing high solubility compounds according to FORMULA I, make it feasible to administer anti-infective therapy in a multi-dosage form, e.g. via an implantable mini-pump (such as used for delivery of insulin in patients with Type 1 insulin-dependent diabetes mellitus).

Embodiments of the invention provide methods for improving the aqueous solubility of poorly soluble pharmaceutical agents. The compositions prepared according to the methods of the invention have improved aqueous solubility. The instant compositions have improved bioavailability providing a pharmacologically effective therapeutic unit dosage at a lower level of administered drug compound. The instant methods thus provide novel formulations and resultant pharmaceutical compositions wherein lower concentrations of pharmaceutical agents provides cost-savings, and at the same time, improvements in efficacy. Bioavailability, in this context, is intended to mean improved pharmacokinetic rates of delivery occassioned e.g., by more effective transport from the gastrointestinal system into blood, or by greater solubility in bodily fluids, as well as, improved stability of drug levels in bodily fluids. In addition to delivery rate improvements, the instant methods provide novel pharmaceutical compositions not previously possible with poorly soluble pharmaceutical agents.

Embodiments of the invention provide treatments for neurologic infections. According to the instant methods, a purpose of therapy in an acute setting may be to rapidly increase the concentration of one or more of the instant composition in a tissue, e.g., by a bolus intravenous injection. Alternatively, in other cases it may desirable to deliver the composition over a longer period of time, e.g., by infusion. The route of delivery according to the instant methods is determined by the infectious disease and the site where treatment is required. For topical application, it may prove desirable to apply the compositions at the local site (e.g., by placing a needle into the tissue at that site) or by placing a timed-release dermal patch); while in a more acute disease clinical setting it may prove desirable to administer the compositions systemically. For other indications the instant compounds may be delivered by intravenous, intraperitoneal, intramuscular, subcutaneous and intradermal injection, as well as, by intranasal and intrabronchial instillation (e.g., with a nebulizer), transdermal delivery (e.g., with a lipid-soluble carrier in a skin patch), or gastrointestinal delivery (e.g., with a capsule or tablet). The preferred therapeutic compositions for inocula and dosage will vary with the clinical indication. The inocula may typically prepared from a dried compound, e.g. by suspending the compound in a physiologically acceptable diluent such as water, saline, or phosphate-buffered saline. Some variation in dosage will necessarily occur depending upon the condition of the patient being treated, and the physician will, in any event, determine the appropriate dose for the individual patient. Since the pharmacokinetics and pharmacodynamics of the instant compounds will vary somewhat in different patients, the most preferred method for achieving a therapeutic concentration in a tissue is to gradually escalate the dosage and monitor the clinical effects. The initial dose, for such an escalating dosage regimen of therapy, will depend upon the route of administration.

In other embodiments, the invention provides methods prophylactic and therapeutic uses in treatment of an infectious disease in a man or domestic animal, involving the step of administering to the subject in need thereof a compound according to FORMULA I, supra. In certain alternative embodiments, the method may involve administration of an intravenous bolus injection or perfusion, or may involve administration during (or after) surgery, or a prophylactic administration. In certain other embodiments, the instant administration may involve a combination therapy, e.g., a compound according to FORMULA I and a second drug, e.g., an anti-coagulant, a second anti-infective agent, an anti-viral agent and/or an anti-hypertensive agent.

The route of delivery of the subject preparations, according to the instant methods, determined by the particular disease. For topical application it may be useful to apply the instant compounds at the local site (e.g., by injection, while for other indications the preparations may be delivered by intravenous, intraperitoneal, intramuscular, subcutaneous, intranasal, and intradermal injection, as well as, by transdermal delivery (e.g., with a lipid-soluble carrier in a skin patch placed on the skin), or even by oral and/or gastrointestinal delivery (e.g., with a capsule, tablet or suppository).

In certain preferred embodiments, the invention provides methods for administering to a subject in need thereof one or more anti-infective agents according to FORMULA I in combination with an agent capable of stimulating intestinal or neural glucose transporter activity, e.g., IGF-1, glucagon, vascular infusions of glucose and the like. The instant combination treatments may be effected by the same route, (e.g., both administered orally), or alternatively, by different routes. Instruction is provided that intestinal glucose saccharide co-transporters exhibit circadian periodicity and expression is inducible by dietary carbohydrate (e.g., see Rhoads et al., 1998), and negatively regulated by leptin (e.g., see Lostao et al. 1998). Thus, in certain embodiments, treatment regimens for oral administration may include instructions to take one or more of the subject compounds orally with a feeding that includes dietary carbohydrate, and preferably, in the morning within about 5 to about 20 minutes after the first meal, and in the evening before,-during or within about 5 to about 20 minutes after an evening meal. Instruction is also provided that during the instant treatment the following are to be avoided because they may alter saccharide transporter activity: namely, (i) high cholesterol diet; (ii) co-administration with oral calcium channel blockers (e.g., see Hyson et al. 1996, 1997); (iii) erythromycin (Navarro et al., 1993); and, (iv) barbiturates (Haspel et al., 1999).

Methods for determining that a test compound according to FORMULA I, i.e., with a drug selected from TABLE A or TABLE B, is suitable for use in one or more of the instant methods, (i.e., for treating infectious disease), are known to those skilled in the art of neuropsychopharmacology, immunology and microbiology. For instance, the test compound may be evaluated in tests in microbiological assays. For example, a representative selection of five pathogenic bacteria may be chosen from among the agents of bacterial meningitis, i.e., Streptococcus pneumoniae, Neisseria meningitidis, Haemophilis influenza, Group B Streptococcus and Escherichia coli (Medical Microbiology, 3^{rd} Edition, Murray, et al, 1998; CDC, http://cdc.gov/ncidod//dbmd). The minimal inhibitory concentration (MIC) of a test compound may be determined on such bacteria by microdilution following established guidelines (National Committee for Clinical Laboratory Standards, 3^{rd} Edition, 1998). Such testing determines the lowest concentration of a test compound capable of preventing visible growth of a bacteria. In addition, it is possible to determine the minimum bactericidal concentration (MBC) of a test compound by counting bacterial colonies growing on microbiological plates, e.g., according to established methods as set forth in guidelines established by the FDA (FDA Bacteriological Analytical Manual, 8^{th} Edition, 1998). In addition, disk-plate-diffusion bioassays of a test compound may be conducted with the size of an inhibitory zone (mm) giving a relative in vitro measure of anti-microbial activity of the subject test compound (Lannette, E. H., Manual of Clinical Microbiology, 4^{th} Edition: American Association for Microbiology, Washington, D.C.). For comparative purposes, it prove worthwhile to compare the activity of a test compound according to the invention with the activity of sulfamethoxizole. In the art, it is appreciated that the combination of MIC value, size of the zone of inhibition in disk-plate-diffusion assays, and MBC are effective in predicting antimicrobial activity of a test compound. Test compounds may also be evaluated in experimental animals; e.g., to determine an ED₅₀, e.g., in rats at an oral dosage of about 0.1-30 mg/kg.

In one embodiment, a pharmaceutical composition for use in humans comprises a therapeutic unit dose effective to delivery to the subject in need thereof about 0.08 mg/kg to about 8 mg/kg of trimethoprim, or a trimethoprim-gluconamide according to FORMULA I, and about 0.04 mg/kg to about 40 mg/kg of a compound according to either of FORMULA IVa or FORMULA IVb per 24 hrs. An illustrative use of the latter composition being therapy in children with otitis media.

In a second embodiment, a pharmaceutical composition for use in humans comprises a therapeutic unit dose effective to delivery to the subject in need thereof about 0.02 mg/kg to about 20 mg/kg of trimethoprim, or trimethoprim-gluconamide according to FORMULA I, and about 1.0 mg/kg to about 100 mg/kg of a compound according to either of FORMULA IVa or FORMULA IVb per 24 hrs. An illustrative use of the latter composition being treatment of patients with pulmonary Pneumocystis carinii infections.

In a third alternative embodiment, a pharmaceutical tablet for use in humans comprises about 1.60 mg to about 160 mg of trimethoprim, or a trimethoprim-gluconamide according to FORMULA I, and about 8 mg to about 800 mg of a compound according to either of FORMULA IVa or FORMULA IVb.

In a fourth embodiment, a pharmaceutical tablet for use in humans comprises about 0.8 mg to about 80 mg of trimethoprim, or a trimethoprim-gluconamide according to FORMULA I, and about 4 mg to about 400 mg of a compound according to either of FORMULA IVa or FORMULA IVb.

The following EXAMPLES illustrate synthetic methods which may prove useful in preparation of the instant N-linked sulfonyl-amine and -amide drugs according to FORMULA I, FORMULA IVa and FORMULA IVb. Additional disclosure of the N-linked glycosyl prodrug pharmaceutical compositions is contained within Applicant's copending U.S. patent application Ser. Nos. 09/547,506 and 09/547,501, both filed on Apr. 12, 2000, and both incorporated herein by reference in their entirety.

### EXAMPLE 1

### Preparation of N-linked Glycosyl Prodrug Compounds: Dopamine Gluconamide and Dopamine Gluconamine

Synthesis of representative alternative N-linked glycosyl prodrug compounds is disclosed in applicants co-pending U.S. patent application Ser. Nos. 09/547,506 and 09/547,501, both filed on Apr. 12, 2000, both incorporated herein by reference in their entirety. Briefly, gluconolactone and 3-hydroxytryamine were reacted slowly in methanol to form a white solid dopamine gluconamide precipitant. The product was collected by filtration, washing and drying in vacuo (i.e., dopamine gluconamide, Compound #1, below).

Synthesis of dopamine gluconamine from the dopamine gluconamide Compound #1 involved first protecting the dopamine aromatic hydroxyl groups by addition of acetone, stirring, refluxing and cooling to form the isopropylidine-protected product as a white solid. The solid was removed by filtration and dried in vacuo. Second, the dopamine gluconamide carbonyl group was reduced by addition of Borane in THF, and after refluxing, cooling, and solvent removal by rotary evaporation methanolic HCl was added and the solution was again refluxed. Solvent was removed by evaporation and the solid dopamine gluconamine product was recrystalized using a mixture of acetonitrile and ethanol. The recrystalized reduced dopamine gluconamine product (i.e., referred to below as Compound #2) was dried in vacuo.

By way of non-limiting illustration, using Applicant's methods amide and amine products were prepared e.g., for at least the following pharmaceutical agents: namely, dopamine ribonamine and ribonamide; p-aminobenzoic acid gluconamine and gluconamide; p-aminosalicyclic acid gluconamine and gluconamide; acyclovir gluconamine and gluconamide; tryptamine gluconamine and gluconamide; sulfamethoxazol gluconamine and gluconamide; sulfasalazine gluconamine and gluconamide; phenethylamine gluconamine and gluconamide; and, benzocaine gluconamine and gluconamide.

### EXAMPLE 2

### Illustrative Ready Solution For Administration as a Measured Dose

Ready solutions for administration as a measured dose were prepared according to TABLE E, below.

TABLE E

| Component | Amount | |
|---|---|---|
| Compound #1 or #2 | 2.5 | gm |
| Methyl-p-aminobenzoic acid | 0.014 | gm |
| Propyl-p-aminobenzoic acid | 0.020 | gm |
| Saccharin sodium | 0.050 | gm |
| Flavoring agent | 0.001 | gm |
| Citric acid | 0.200 | gm |
| Sodium citrate | 0.320 | gm |
| Distilled water USP q.s. to | 100 | ml |

### EXAMPLE 3

### Illustrative Powder Composition for Reconstitution Prior to Use

Powder composition suitable for reconstitution before use were prepared according to TABLE F.

TABLE F

| Component | Amount | |
|---|---|---|
| Compound #1 or #2 | 2.5 | mg |
| Sodium citrate | 20.0 | mg |
| Sorbitol | 2.0 | mg |
| Flavoring agent | 0.1 | mg |
| Distilled water USP for | 10.0 | ml |
| reconstitution | | |

### EXAMPLE 4

### Illustrative Tablets for Oral Administration

Tablets for oral administration were prepared according to TABLE G.

TABLE G

| Component | Amount |
|---|---|
| Compound #1 or #2 | 250 mg |
| Starch | 17 mg |
| Sodium glycolate (starch) | 40 mg |
| Polyvinal pyrrolidene | 7.0 mg |
| Microcrystalline cellulose | 45 mg |
| Magnesium sterate | 2.0 mg |

### EXAMPLE 5

### Illustrateive Tablets for Sublingual Administration

Tablets for sublingual administration were prepared according to TABLE H.

TABLE H

| Component | Amount |
|---|---|
| Compound #1 or #2 | 250 mg |
| Gum arabic | 10 mg |
| Lactose | 90 mg |
| Ammonium glycyrrhiznate | 20 mg |
| Sodium saccharin | 2 mg |
| Flavor | 10 mg |
| Magnesium sterate | 7 mg |

### EXAMPLE 6

### Preparation of N-linked Glycosyl Prodrug Compounds: Preparation of Sulfamethoxazole Gluconamide-1

Sulfamethoxazole (1.42 g, 5.6 mmol) was added with stirring to a 100 mL round bottom flask containing δ-gluconolactone (1.00 gm, 5.6 mmol), triethylamine (5.7 gm, 5.6 mmol) and 30 mL of acetonitrile. The mixture as refluxed for 4.5 hr. during which time the reaction mixture became homogeneous. After allowing the solution to cool to room temperature, solvent was removed by rotary evaporation. The resultant reaction product was rinsed with cold acetonitrile to give a white solid sulfamethoxazole gluconamide (1.36 gm, about 56.2% yield) having a melting point of 154-155° C.

Analysis Calculated for C₁₆H₂₁N₃O₉S₁ (431.42): C, 44.55; H, 4.91; N, 9.74; S, 7.43. Found: C, 44.6; H, 4.91; N, 9.73; S, 7.34.

### EXAMPLE 7

### Preparation of N-linked Glycosyl Prodrug Compounds: Preparation of Sulfamethoxazole Gluconamide-2

Alternatively, sulfamethoxazole (1.42 g, 5.6 mmol) was added with stirring to a 100 mL round bottom flask containing δ-gluconolactone (1.00 gm, 5.6 mmol) and 30 mL of methanol. The mixture as refluxed for 4.5 hr. during which time the reaction mixture became homogeneous. After allowing the solution to cool to room temperature, solvent was removed and the product collected by filtration. The resultant reaction product was rinsed with cold acetonitrile to give a white solid sulfamethoxazole gluconamide (1.36 gm, about 60% yield) having a melting point of 154-155° C.

Analysis Calculated for C₁₆H₂₁N₃O₉S₁ (431.42): C, 44.55; H, 4.91; N; 9.74; S, 7.43. Found: C, 44.6; H, 4.91; N, 9.73; S, 7.34.

### Citations

Bodor et al., 1978. J. Pharm. Sci, 67 (5): 685.

Bodor, 1976. “Novel Approaches for the Design of Membrane Transport Properties of Drugs”. In: “Design of Biopharmaceutical Properties Through Prodrugs and Analogs”, Ed. E. B. Roche et al. APhA Academy of Pharmaceutical Sciences, Washington, D.C., pp. 98-135

Bodor et al, 1981. Science 214: 1370-1372.

Bodor et al, 1983. Pharmacology and Therapeutics 19 (3): 337-386.

Curtis, B. R., McFarland, J. G., Guo-Guang, W., Visentin, G. P. and R. H. Aster. 1994. Antibodies in sulfonamide-induced immune thrombocytopenia recognize calcium-dependent epitopes on the glycoprotein IIb/IIIa complex. Blood 84 (1): 176-183.

Duport, S., Robert, F., Muller, D., Grau, G., Parisi, L. and L. Stoppini. 1998. An in vitro blood-brain barrier model: Cocultures between endothelial cells and organotypic brain slice cultures. Proc. Natl. Acad. Sci. USA 95 (4): 1840-1845.

Findlay, J., Levy, G. A. and C. A. Marsh. 1958. Inhibition of glycosidases by aldonolactones or corresponding configuration. 2. Inhibitors of β-N-acetylglucosaminidase. Biochemical J. 69: 467-476.

Fodor et al. 1961. Acta Chim. Acad. Sci. Hung. 28 (4): 409.

Gee, J. M., DuPont, M. S., Rhodes, M. J. and I. T. Johnson. 1998. Quercetin glucosides interact with the intestinal glucose transporter pathway. Free Radic. Biol. Med. 25 (1): 19-25.

Green, M. D. and T. R. Tephly. 1996. Glucuronidation of amines and hydroxylated xenobiotics and endobiotics catalyzed by expressed human UGT1.4 protein. Drug Metab. Dispos. 24 (3): 356-363.

Haspel, H. C., Stephenson, K. N., Davies-Hill, T., El-Barbary, A., Lobo, J. F., Croxen, R. L., Mougrabi, W., Koehler-Stec, E. M., Fenstermacher, J. D. and I. A. Simpson. 1999. Effects of barbiturates on facilitative glucose transporters are pharmacologically specific and isoform selective. J. Membr. Biol. 169 (1): 45-53.

Horton, D. 1969. Monosaccharide Amino Sugars. In: “The Amino Sugars”: The Chemistry and Biology of Compounds Containing Amino Sugars. Vol. 1A. Ed. R. W. Jeanloz. Academic Press, N.Y. pp. 4-18.

Huang, X., Xu, R., Hawley, M. D., Hopkins, T. L. and K. J. Kramer. 1998. Electrochemical oxidation of N-acyldopamines and regioselective reactions of their quinones with N-acetylcysteine and thiourea. Arch. Biochem. Biophys. 352 (1): 19-30.

Hyson, D. H., Thomson, A. B. and C. T. Kappagoda. 1996. Calcium channel blockers modify jejunal uptake of D-galactose in rabbits. Dig. Dis. Sci. 41 (9): 1871-1875.

Hyson, D. H., Thomson, A. B., Keelan, M. and C. T. Kappagoda. 1997. A high cholesterol diet blocks the effect of calcium channel blockers on the uptake of sugars in rabbit intestine. Can. J. Physiol. Pharmacol. 75 (1): 57-64.

Kerwin, J. L. 1996. Negative ion electrospray mass spectrometry of polyphenols, catecholamines and their oxidation products. J. Mass Sprectrom. 31: 1429-1439.

Kerwin, J. L. 1997. Profiling peptide adducts of oxidized N-acetyldopamine by electrospray mass spectrometry. Rapid Commun. Mass Sprectrom. 11: 557-566.

Kerwin, J. L., Whitney, D. L. and A. Sheikh. 1999. Mass spectrometry of glucosamine, glucosamine polymers and their catecholamine adducts. Model reactions and cuticular hydrolysates of Toxorhynchites amboinensis (Culicidae) pupae. Insect Biochem. Mol. Biol. 29 (7): 599-607.

Kumagai, A. K. 1999. Glucose transport in brain and retina: Implications in the management and complications of diabetes. Diabetes Metab. Res. Rev. 15 (4): 261-273.

Lostao, M. P., Urdaneta, E., Martinez-Anso, E., Barber, A. and J. A. Martinez. 1998. Presence of leptin receptors in rat small intestine and leptin effect on sugar absorption. FEBS Lett. 423 (3): 302-306.

Manzi, A. E. and A. Varki. 1993. In: Glycobiology: A Practical Approach. Eds. M. Fukuda and A. Kobata. IRL Press, Oxford University, Oxford. pp 29-31.

Martin, M. G., Turk, E., Lostao, M. P., Kerner, C. and E. M. Wright. 1996. Defects in Na+/glucose cotransporter (SGLT1) trafficking and function cause glucose-galactose malabsorption. Nat. Genet. 12 (2): 216-220.

Mauri-Hellwig, D., Bettens, F., Mauri, D., Brander, C., Hunziker, T. and W. J. Pichler. 1995. Activation of drug-specific CD4+ and CD8+ T cells in individuals allergic to sulfonamides, phenytoin and carbamazepine. J. Immunool. 155: 462.

Mizuma, T., Ohta, K. and S. Awazu. 1994. The beta-anomeric and glucose preferences of glucose transport carrier for intestinal active absorption of monosaccharide conjugates. Biochim. Biophys. Acta 1200 (2): 117-122.

Navarro, H., Arruebo, M. P., Alcalde, A. I. and V. Sorribas. 1993. Effect of erythromycin on D-galactose absorption and sucrase activity in rabbit jejunum. Can. J. Physiol. Pharmacol. 71 (3-4): 191-194.

Rhoads, D. B., Rosenbaum, D. H., Unsal, H., Isselbacher, K. J. and L. L. Levitsky. 1998. Circadian periodicity of intestinal Na+/glucose cotransporter 1 mRNA levels is transcriptionally regulated. J. Biol. Chem. 273 (16): 9510-9516.

Schauer, R. 1978. In: Methods in Enzymology, Ed. V. Ginsberg. Academic Press, NY. pp. 64-89.

Vannucci, S. J., Clark, R. R., Koehler-Stec, E., Li, K., Smith, C. B., Davies, P., Maher, F. and I. A. Simpson. 1998. Glucose transporter expression in brain: Relationship to cerebral glucose utilization. Dev. Neurosci. 20 (4-5): 369-379.

von Greyerz, S., Zanni, M. P., Frutig, K., Schnyder, B., Burkhart, C. and W. J. Pichler. 1999. Interaction of sulfonamide derivatives with the TCR of sulfamethoxazole-specific human αβ+ T cell clones. J. Immunology 162: 595-602.

Wright, E. M., Hirsch, J. R., Loo, D. D. and G. A. Zampighi. 1997. Regulation of Na+/glucose cotransporters. J. Exp. Biol. 200 (2): 287-293.

While the preferred embodiment of the invention has been illustrated and described, it will be appreciated that various changes can be made therein without departing from the spirit and scope of the invention.

I claim:

1. A pharmaceutical composition for neuraxial delivery comprising both a hydrophilic N-linked glycosyl prodrug compound and a formulary, wherein said hydrophilic N-linked glycosyl prodrug compound comprises an anti-infective prodrug compound covalently linked with a saccharide through an amide or an amine bond and said formulary comprises an agent selected from the group consisting of an additive, a stabilizer, a carrier, a binder, a buffer, an excipient, an emollient, a disintegrant, a lubricating agent, an antimicrobial agent and a preservative,
with the proviso that said saccharide moiety is not a cyclodextrin or a glucuronide.

2. The pharmaceutical composition of claim 1, further comprising a dosage form selected from the group consisting of a powder, a granule, an emollient cream, a tablet, a capsule, a lozenge, a trouch, a suppository, a perenteral solution, an injection solution, a syrup, an elixir, a nasal solution, a intrabronchial solution, an ophthalmic solution, a dermal patch and a bandage.

3. The pharmaceutical composition of claim 1, wherein said hydrophilic N-linked glycosyl prodrug compound further comprises a compound according to FORMULA I:
A—B—D—E
Formula I
wherein, each of “—” comprises a single bond; A, comprises an anti-infective prodrug compound; B, comprises a lower alkyl; D, comprises a nitrogen linker amine or amide; and, E comprises a saccharide, with the proviso that E is not a cyclodextrin or a glucuronide.

4. The pharmaceutical composition of claim 3 wherein said A-moiety comprises a sulfonamide anti-infective prodrug compound.

5. The pharmaceutical composition of claim 4, wherein said sulfonamide is selected from the group consisting of sulphamethoxazole, sulphamerazine, sulfathiazole, sulfatroxazole, sulfadiazine, sulfasalezine, sulfadimethoxine and sulfapyridine.

6. The pharmaceutical composition of claim 5, wherein said anti-infective prodrug comprises sulfamethoxazole.

7. A process for preparing a hydrophilic N-linked glycosyl prodrug compound for neuraxial delivery, comprising the step of reacting an anti-infective prodrug compound with a saccharide moiety under conditions suitable for formation of an amide or amine bond between said anti-infective prodrug compound and said saccharide moiety.

8. The process of claim 7, wherein said hydrophilic N-linked glycosyl prodrug compound comprises a compound according to FORMULA I:
A—B—D—E
Formula I
wherein, each of “—” comprises a single bond; A, comprises said anti-infective prodrug; B, comprises an optional lower alkyl; D, comprises said N-linker amine or amide; and, E comprises said saccharide, with the proviso that E is not a cyclodextrin or a glucuronide.

9. A process for preparing a pharmaceutical composition comprising hydrophilic N-linked glycosyl prodrug compound for neuraxial delivery, comprising the steps of reacting an anti-infective prodrug compound with a saccharide moiety under conditions suitable for formation of an amide or amine bond between said anti-infective prodrug compound and said saccharide moiety; and formulating said N-linked glycosyl prodrug compound into said pharmaceutical composition by addition of an agent selected from the group consisting of an additive, a stabilizer, a carrier, a binder, a buffer, an excipient, an emollient, a disintegrant, a lubricating agent, an antimicrobial agent and a preservative.

10. A method for treating a neurological infection in a subject in need thereof comprising the step of administering to the subject a pharmaceutical composition comprising a compound according to FORMULA I:
A—B—D—E
Formula I
wherein, each of “—” comprises a single bond; A, comprises an anti-infective prodrug; B, comprises a lower alkyl; D, comprises a nitrogen linker amine or amide; and, E comprises a saccharide, with the proviso that E is not a cyclodextrin.

11. A pharmaceutical composition comprising an anti-infective prodrug compound according to either of FORMULA IVa or FORMULA IVb, 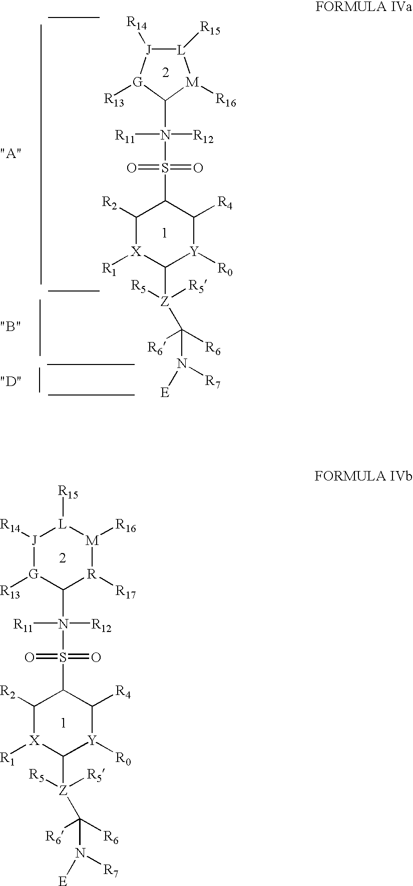
wherein, Ring 1 and Ring 2 each independently comprise an optionally substituted cyclic or heterocyclic ring, or an optionally substituted aromatic ring, Ring 1 comprising about 4 to about 8 carbon atoms, among which are counted “X” and “Y” and Ring 2 comprising about 4 to about 6 atoms, among which are counted “G”, “J”, “L”, “Q” and optional ring components “M” and “R”; the constituent atoms of Ring 2 being selected according to either of TABLE C or TABLE D; “X” and “Y” each comprise a carbon atom;
Z is optional and when present comprises a lower alkyl optionally substituted with R₅ and R_{5′};
N comprises a nitrogen atom of a primary or secondary amine or an amide and R₇ comprises hydrogen or methyl,
E comprises a saccharide moiety;
R₀, R₁, R₂, R₃ and R₄ each independently comprise a group selected from among hydrogen, hydroxyl, halogen, halo-lower alkyl, alkoxy, alkoxy-lower alkyl, halo-alkoxy, thioamido, amidosulfonyl, alkoxycarbonyl, carboxamide, amino-carbonyl, and alkylamine-carbonyl; and,
R₁₃, R₁₄, R₁₅, R₁₆ each independently comprise a group selected from among hydrogen, hydroxyl, lower alkyl and alkoxyl-lower alkyl.

12. The anti-infective pharmaceutical composition of claim 11, wherein Z is absent or a lower alkyl comprising 1 or 2 carbon atoms.

13. The anti-infective pharmaceutical composition according to claim 12, wherein Z is absent or a one carbon atom.

14. The anti-infective pharmaceutical composition of claim 11, wherein each of R₅ and R₅, when present, and each of R₆ and R_{6′}, when present, independently comprise a group selected from among hydrogen, hydroxyl, alkoxyl, carboxyl, alkoxylcarbonyl, aminocarbonyl, alkylamino-carbonyl and dialkylamino-carbonyl.

15. The anti-infective pharmaceutical composition of claim 11, wherein R₇ comprised a hydrogen atom.

16. The anti-infective pharmaceutical composition of claim 11, wherein R₀, R₁, R₂, R₃ and R₄ each independently comprise a group selected from hydrogen, hydroxyl, lower alkyl and alkoxyl-lower alkyl.

17. The anti-infective pharmaceutical composition of claim 16, wherein R₀, R₁, R₂, R₃ and R₄ each independently comprise hydrogen.

18. The anti-infective pharmaceutical composition of claim 11, wherein Ring 2 comprises a 5-membered ring.

19. The anti-infective pharmaceutical composition of claim 11, wherein Ring 2 comprises an aryl or heteroaryl ring.

20. The anti-infective pharmaceutical composition of claim 11, wherein R₁₃, R₁₄, R₁₅, R₁₆ each independently comprise hydrogen or lower alkyl.

21. The anti-infective pharmaceutical composition of claim 11, wherein R₁₃, R₁₄, R₁₅, R₁₆ each independently comprise hydrogen or lower alkyl.

22. The anti-infective pharmaceutical composition of claim 11, wherein said R₅ and R_{5′} are selected from the group consisting of hydrogen, hydroxyl, alkoxyl, carboxyl, alkoxylcarbonyl, aminocarbonyl, alkylamino-carbonyl and dialkylamino-carbonyl.

23. The anti-infective pharmaceutical composition of claim 11, wherein said E substituent is selected from the group consisting of a radical of a monosaccharide, a disaccharide, a trisaccharide and an oligosaccharide

24. The anti-infective pharmaceutical composition of claim 1, wherein said E monosaccharide comprises a radical of a sugar selected from the group consisting of aldose, ketoaldose, alditols, ketoses, aldonic acids, ketoaldonic acids, aldaric acids, ketoaldaric acids, amino sugars, keto-amino sugars, uronic acids, ketouronic acids, lactones and keto-lactones.

25. The anti-infective pharmaceutical composition of claim 24, wherein said radical of a sugar is further selected from the group consisting of triosyl, tetraosyl, pentosyl, hexosyl, heptosyl, octosyl and nonosyl radicals and derivatives thereof.

26. The anti-infective pharmaceutical composition of claim 25, wherein said pentosyl sugar radical comprises a straight carbon chain, a furanosyl ring or a derivative thereof.

27. The anti-infective pharmaceutical composition of claim 25, wherein said hexosyl sugar radical comprises a straight carbon chain, a furanosyl ring, a pyranosyl ring or a derivative thereof.

28. The anti-infective pharmaceutical composition of claim 25, wherein said hexosyl radical is further selected from the group consisting of allose, altrose, glucose, mannose, gulose, idose, galactose, talose, fructose, ribo-hexulose, arabino-hexulose, lyxo-hexulose and derivatives thereof.

29. The anti-infective pharmaceutical composition of claim 25, wherein said pentosyl radical is further selected from the group consisting of ribose, arabinose, xylose, lyxose, ribulose, xylulose and derivatives thereof.

30. The anti-infective pharmaceutical composition of claim 25, wherein said heptosyl residue comprises sedoheptulose and derivatives thereof.

31. The anti-infective pharmaceutical composition of claim 25, wherein said nonosyl residue comprises N-acetylneuraminic acid, N-glycolylneuraminic acid, diacetylneuranminic acid, and derivatives thereof.

32. The anti-infective pharmaceutical composition of claim 28, wherein said compound further comprises glucose, galactose, fructose or derivatives thereof.

33. The anti-infective pharmaceutical composition of claim 23, wherein said disaccharide, trisaccharide and oligosaccharide comprise a sugar homopolymer or a sugar heteropolymer.

34. The anti-infective pharmaceutical composition of claim 33, wherein said sugar homopolymer comprises a glycoside selected from the group consisting of erythran, threan, riban, arabinan, xylan, lyxan, allan, altran, glucan, mannan, gulan, idan, galactan, talan, fructan and derivatives thereof.

35. The anti-infective pharmaceutical composition of claim 33, wherein said sugar heteropolymer further comprises a glycoside selected from the group consisting of erythroside, threoside, riboside, arabinoside, xyloside, lyxoside, alloside, altroside, glucoside, mannoside, guloside, idoside, galactoside, taloside, fructoside and derivatives thereof.

36. The anti-infective pharmaceutical composition of claim 35, wherein said sugar heteropolymer further comprises a glycoside metabolized in a mammal to a glucosyl or a galactosyl monosaccharide.

37. The anti-infective pharmaceutical composition of claim 34, wherein said glycoside further comprises a riban, an arabinan, a glucan, a galactan, a mannan and derivatives thereof.

38. The anti-infective pharmaceutical composition of claim 35, wherein said glycoside further comprises a riboside, an arabinoside, a glucoside, a galactoside, a mannoside, a fructoside and derivatives thereof.

39. The anti-infective pharmaceutical composition of claim 36, wherein said glucan comprises maltose, amylose, glycogen, cellobiose, amylopectin, heparin and derivatives thereof.

40. The anti-infective pharmaceutical composition of claim 37, wherein said glucoside comprises sucrose and derivatives thereof.

41. The anti-infective pharmaceutical composition of claim 37, wherein said fructoside comprises fucosidolactose and derivatives thereof.

42. The anti-infective pharmaceutical composition of claim 37, wherein said galactoside comprises lactose, hyaluronic acid, pectin and derivatives thereof.

43. A method for improving the aqueous solubility and blood brain barrier penetrability of a drug, comprising the step of forming a covalent chemical bond between the drug and a sugar or oligosaccharide, wherein said drug comprises an amide or amine group and said drug bonded to said sugar or oligosaccharide comprises a compound according to FORMULA I:
A—B—D—E
Formula I
wherein, each of “—” comprises a single bond; A, comprises a Anti-infective prodrug; B, comprises a lower alkyl; D, comprises a nitrogen linker amine or amide; and, E comprises a saccharide, with the proviso that E is not a cyclodextrin.
